(19) United States
(12) Patent Application Publication
(10) Pub. No.: US 2017/0370167 A1
(43) Pub. Date: Dec. 28, 2017

(54) MUD-GAS SEPARATOR APPARATUS AND METHODS

(71) Applicant: SEABOARD INTERNATIONAL INC., Houston, TX (US)

(72) Inventors: LEVENT AKTAS, NORMAN, OK (US); TIMOTHY LONG, OKLAHOMA CITY, OK (US); MATTHEW B. GREEN, NORMAN, OK (US); JOHN MATHENA, EDMOND, OK (US)

(73) Assignee: SEABOARD INTERNATIONAL INC., Houston, TX (US)

(21) Appl. No.: 15/533,597

(22) PCT Filed: Dec. 9, 2015
(86) PCT No.: PCT/US15/64625

§ 371 Date: Jun. 6, 2017

Related U.S. Application Data
(60) Provisional application No. 62/089,913, filed on Dec. 10, 2014.
(60) Provisional application No. 62/173,633, filed on Jun. 10, 2015.

(51) Int. Cl. E21B 21/06; G01F 23/296; G01N 33/28; E21B 21/08; G01F 25/00; E21B 49/10; E21B 44/06

(52) U.S. Cl. CPC E21B 21/067; G01N 33/2823; G01F 23/2967; E21B 21/08; E21B 49/10; E21B 44/06; G01F 25/0007

(57) ABSTRACT

In one aspect, a mud-gas separator vessel defines an internal region in which a slurry is adapted to be collected. The slurry defines a fluid level. A sensor is adapted to measure the fluid level. An electronic controller is in communication with the sensor and is adapted to receive measurement data. A control valve is in communication with the controller and is adapted to control discharge of the slurry. The controller is adapted to automatically control the control valve based on the measurement data and thus actively control the fluid level using the control valve. In another aspect, a method is provided for automatically maintaining the fluid level to prevent vent gas carry under from the separator vessel. In another aspect, a kit is provided for actively controlling the fluid level. In another aspect, a method of retrofitting a mud-gas separator apparatus is provided.

### CROSS-REFERENCE TO RELATED APPLICATIONS

This application claims the benefit of the filing date of, and priority to, U.S. patent application No. 62/089,913, filed Dec. 10, 2014, the entire disclosure of which is hereby incorporated herein by reference.

This application claims the benefit of the filing date of, and priority to, U.S. patent application No. 62/173,633, filed Jun. 10, 2015, the entire disclosure of which is hereby incorporated herein by reference.

### TECHNICAL FIELD

This disclosure relates in general to mud-gas separators and, in particular, to automatically controlling one or more aspects of a mud-gas separator apparatus.

### BACKGROUND OF THE DISCLOSURE

During the drilling of an oil or gas well, different materials may be discharged from the well. The discharged materials may include mixtures of solid, liquid, and gas materials. A mud-gas separator may be used to separate the gas materials from the solid and liquid materials. After separation, the gas materials flow out through a gas vent line, and the solid and liquid materials flow out through a slurry return line. In some cases, a mud-gas separator apparatus may have too large of a footprint, taking up too much ground space at a wellsite. Also, the mud-gas separator apparatus may have too large of a volume, taking up too much volumetric space during transportation to the wellsite and/or during operation at the wellsite. Further, the gas materials may flow out of the slurry return line, rather than out of the gas vent line, increasing the risk of fire at the wellsite. Still further, personnel at the wellsite may not be aware that the amount of solid and liquid materials in the mud-gas separator vessel at any given time is either too high or too low. Therefore, what is needed is an apparatus, method, or kit that addresses one or more of the foregoing issues, or other issue(s).

### SUMMARY

In a first aspect, there is provided an apparatus that includes a mud-gas separator vessel adapted to receive a multiphase flow and separate gas materials therefrom. The mud-gas separator vessel defines an internal region in which a slurry is adapted to be collected, and the slurry defines a fluid level within the internal region. At least one sensor is operably coupled to the mud-gas separator vessel and is adapted to measure the fluid level when the slurry is collected in the internal region. An electronic controller is in communication with the at least one sensor and is adapted to receive from the at least one sensor measurement data associated with the measurement of the fluid level. A control valve is in communication with the electronic controller and is adapted to control discharge of the slurry out of the mud-gas separator vessel. The electronic controller is adapted to automatically control the control valve based on the measurement data received from the at least one sensor and thus actively control the fluid level within the internal region using the control valve.

In an exemplary embodiment, the control valve includes an electric actuator and a rotary control valve operably coupled thereto.

In another exemplary embodiment, the at least one sensor includes a guided wave level sensor, the guided wave level sensor including a probe, and the apparatus further includes a level sensor housing assembly connected to the mud-gas separator vessel, the level sensor housing assembly including a tubular member within which at least a portion of the probe extends.

In yet another exemplary embodiment, the level sensor housing assembly further includes first and second fittings between which the tubular member extends; and first and second isolation valves connected to the first and second fittings, respectively, and to the mud-gas separator vessel; wherein the tubular member is spaced from the mud-gas separator vessel; and wherein the guided wave level sensor is connected to the second fitting and the probe extends through the second fitting and at least into the tubular member.

In certain exemplary embodiments, the electronic controller includes one or more processors; a non-transitory computer readable medium operably coupled to the one or more processors; and a plurality of instructions stored on the non-transitory computer readable medium and executable by the one or more processors, the plurality of instructions including instructions that cause the one or more processors to automatically control the control valve based on the measurement data.

In an exemplary embodiment, the instructions that cause the one or more processors to automatically control the control valve include instructions that cause the one or more processors to automatically further close the control valve in response to determining that the fluid level is decreasing too rapidly; and instructions that cause the one or more processors to automatically open, or further open, the control valve in response to determining that the fluid level is increasing too rapidly.

In another exemplary embodiment, the instructions that cause the one or more processors to automatically control the control valve include instructions that cause the one or more processors to determine that the fluid level is not within a stability zone; and instructions that cause the one or more processors to automatically adjust a valve position of the control valve in response to determining that the fluid level is not within the stability zone.

In yet another exemplary embodiment, the instructions that cause the one or more processors to automatically control the control valve include instructions that cause the one or more processors to determine a proportional parameter; and instructions that cause the one or more processors to determine a differential parameter.

In still yet another exemplary embodiment, the instructions that cause the one or more processors to automatically control the control valve further include instructions that cause the one or more processors to determine a valve position change based on the proportional and differential parameters if either: the proportional parameter is not less than a proportional fluctuation constant, or the differential parameter is not less than a differential fluctuation constant; and instructions that cause the one or more processors to set a change in a valve position of the control valve to zero degrees if: the proportional parameter is less than the proportional fluctuation constant, and the differential parameter is less than the differential fluctuation constant.

In certain exemplary embodiments, the instructions that cause the one or more processors to automatically control the control valve further include instructions that cause the one or more processors to determine a maximum allowable valve position change if a rate of change of the valve position due to the valve position change based on the proportional and differential parameters would not be less than an allowable angular velocity of the control valve.

In an exemplary embodiment, the instructions that cause the one or more processors to automatically control the control valve further include instructions that cause the one or more processors to update the valve position of the control valve by the valve position change based on the proportional and differential parameters if: the rate of change of the valve position due to the valve position change based on the proportional and differential parameters would be less than the allowable angular velocity of the control valve; and either: the proportional parameter is not less than the proportional fluctuation constant, or the differential parameter is not less than the differential fluctuation constant; instructions that cause the one or more processors to update the valve position of the control valve by the maximum allowable valve position change if: the rate of change of the valve position due to the valve position change based on the proportional and differential parameters would not be less than the allowable angular velocity of the control valve; and either: the proportional parameter is not less than the proportional fluctuation constant, or the differential parameter is not less than the differential fluctuation constant; and instructions that cause the one or more processors to update the valve position of the control valve by zero degrees if: the proportional parameter is less than a proportional fluctuation constant; and the differential parameter is less than a differential fluctuation constant.

In a second aspect, there is provided a method of actively controlling a fluid level in an internal region defined by a mud-gas separator vessel, the fluid level being defined by a slurry collected within the internal region, the method including automatically measuring, using at least one sensor, the fluid level in the internal region; automatically transmitting, using the at least one sensor, measurement data to an electronic controller, the measurement data being associated with the measurement of the fluid level defined by the slurry; and automatically controlling, using the electronic controller, a control valve based on the measurement data; wherein the automatic control of the control valve by the electronic controller automatically controls discharge of the slurry out of the mud-gas separator vessel and thus actively controls the fluid level.

In an exemplary embodiment, automatically controlling the control valve includes automatically further closing the control valve in response to determining that the fluid level is decreasing too rapidly; and automatically opening, or further opening, the control valve in response to determining that the fluid level is increasing too rapidly.

In another exemplary embodiment, automatically controlling the control valve includes automatically determining that the fluid level is not within a stability zone; and automatically adjusting the valve position of the control valve in response to determining that the fluid level is not within the stability zone.

In yet another exemplary embodiment, automatically controlling the control valve includes automatically determining a proportional parameter; and automatically determining a differential parameter.

In still yet another exemplary embodiment, automatically controlling the control valve further includes automatically determining a valve position change based on the proportional and differential parameters if either: the proportional parameter is not less than a proportional fluctuation constant, or the differential parameter is not less than a differential fluctuation constant; and automatically setting a change in a valve position of the control valve to zero degrees if: the proportional parameter is less than the proportional fluctuation constant, and the differential parameter is less than the differential fluctuation constant.

In certain exemplary embodiments, automatically controlling the control valve further includes automatically determining a maximum allowable valve position change if a rate of change of the valve position due to the valve position change based on the proportional and differential parameters would not be less than an allowable angular velocity of the control valve.

In an exemplary embodiment, automatically controlling the control valve further includes automatically updating the valve position of the control valve by the valve position change based on the proportional and differential parameters if: the rate of change of the valve position due to the valve position change based on the proportional and differential parameters would be less than the allowable angular velocity of the control valve; and either: the proportional parameter is not less than the proportional fluctuation constant, or the differential parameter is not less than the differential fluctuation constant; automatically updating the valve position of the control valve by the maximum allowable valve position change if: the rate of change of the valve position due to the valve position change based on the proportional and differential parameters would not be less than the allowable angular velocity of the control valve; and either: the proportional parameter is not less than the proportional fluctuation constant, or the differential parameter is not less than the differential fluctuation constant; and automatically updating the valve position of the control valve by zero degrees if: the proportional parameter is less than the proportional fluctuation constant; and the differential parameter is less than the differential fluctuation constant.

In a third aspect, there is provided a method of retrofitting a mud-gas separator apparatus, the mud-gas separator apparatus including a mud-gas separator vessel and a slurry return line connected thereto, the method including operably coupling at least one sensor to the mud-gas separator vessel; operably coupling an electronic controller to the at least one sensor; operably coupling a control valve to the electronic controller; and connecting the control valve to the slurry return line.

In another exemplary embodiment, operably coupling the at least one sensor to the mud-gas separator vessel includes operably coupling a guided wave level sensor to the mud-gas separator vessel.

In yet another exemplary embodiment, operably coupling the guided wave level sensor to the mud-gas separator vessel includes connecting the guided wave level sensor to a level sensor housing assembly; and connecting the level sensor housing assembly to the mud-gas separator vessel.

In certain exemplary embodiments, the guided wave level sensor includes a probe; wherein the level sensor housing assembly includes a tubular member; first and second fittings between which the tubular member extends; and first and second isolation valves connected to the first and second fittings, respectively; wherein connecting the guided wave level sensor to the level sensor housing assembly includes inserting the probe through the second fitting and into at least the tubular member; and connecting the guided wave level sensor to the second fitting; and wherein connecting the level sensor housing assembly to the mud-gas separator vessel includes connecting the first and second isolation valves to the mud-gas separator vessel so that the tubular member is spaced from the mud-gas separator vessel.

In an exemplary embodiment, operably coupling the control valve to the electronic controller includes operably coupling an electric actuator to the electronic controller; and operably coupling a rotary control valve to the electric actuator.

In another exemplary embodiment, the mud-gas separator vessel defines an internal region; wherein the at least one sensor is adapted to measure the fluid level when a slurry is collected in the internal region; wherein the control valve is adapted to control discharge of the slurry out of the mud-gas separator vessel; and wherein the electronic controller is adapted to receive from the at least one sensor measurement data associated with the measurement of the fluid level, and is further adapted to automatically control the control valve based on the measurement data and thus actively control the fluid level within the internal region using the control valve.

In a fourth aspect, there is provided a kit for actively controlling a fluid level within an internal region defined by a mud-gas separator vessel, the fluid level being defined by a slurry collected within the internal region, the kit including at least one sensor adapted to be operably coupled to the mud-gas separator vessel, and to measure the fluid level when the slurry is collected in the internal region; an electronic controller adapted to be in communication with the at least one sensor, and to receive from the at least one sensor measurement data associated with the measurement of the fluid level; and a control valve adapted to be in communication with the electronic controller, and to control discharge of the slurry out of the mud-gas separator vessel; wherein the electronic controller is adapted to automatically control the control valve based on the measurement data received from the at least one sensor and thus is adapted to actively control the fluid level within the internal region using the control valve.

In an exemplary embodiment, the at least one sensor includes a guided wave level sensor, the guided wave level sensor including a probe; and wherein the kit further includes a level sensor housing assembly adapted to be connected to the mud-gas separator vessel, the level sensor housing assembly including a tubular member within which at least a portion of the probe extends.

In another exemplary embodiment, the level sensor housing assembly further includes first and second fittings between which the tubular member extends; and first and second isolation valves connected to the first and second fittings, respectively, and adapted to be connected to the mud-gas separator vessel; wherein the guided wave level sensor is connected to the second fitting and the probe extends through the second fitting and at least into the tubular member.

In yet another exemplary embodiment, the electronic controller includes one or more processors; a non-transitory computer readable medium operably coupled to the one or more processors; and a plurality of instructions stored on the non-transitory computer readable medium and executable by the one or more processors, the plurality of instructions including instructions that cause the one or more processors to automatically control the control valve based on the measurement data.

In certain exemplary embodiments, the instructions that cause the one or more processors to automatically control the control valve include instructions that cause the one or more processors to automatically further close the control valve in response to determining that the fluid level is decreasing too rapidly; and instructions that cause the one or more processors to automatically open, or further open, the control valve in response to determining that the fluid level is increasing too rapidly.

In an exemplary embodiment, the instructions that cause the one or more processors to automatically control the control valve include instructions that cause the one or more processors to determine that the fluid level is not within a stability zone; and instructions that cause the one or more processors to automatically adjust a valve position of the control valve in response to determining that the fluid level is not within the stability zone.

In another exemplary embodiment, the instructions that cause the one or more processors to automatically control the control valve include instructions that cause the one or more processors to determine a proportional parameter; and instructions that cause the one or more processors to determine a differential parameter.

In yet another exemplary embodiment, the instructions that cause the one or more processors to automatically control the control valve include instructions that cause the one or more processors to determine a valve position change based on the proportional and differential parameters if either: the proportional parameter is not less than a proportional fluctuation constant, or the differential parameter is not less than a differential fluctuation constant; and instructions that cause the one or more processors to set a change in a valve position of the control valve to zero degrees if: the proportional parameter is less than the proportional fluctuation constant, and the differential parameter is less than the differential fluctuation constant.

In still yet another exemplary embodiment, the instructions that cause the one or more processors to automatically control the control valve further include instructions that cause the one or more processors to determine a maximum allowable valve position change if a rate of change of the valve position due to the valve position change based on the proportional and differential parameters is not less than an allowable angular velocity of the control valve.

In certain exemplary embodiment, the instructions that cause the one or more processors to automatically control the control valve further include instructions that cause the one or more processors to update the valve position of the control valve by the valve position change based on the proportional and differential parameters if: the rate of change of the valve position due to the valve position change based on the proportional and differential parameters would be less than the allowable angular velocity of the control valve; and either: the proportional parameter is not less than the proportional fluctuation constant, or the differential parameter is not less than the differential fluctuation constant; instructions that cause the one or more processors to update the valve position of the control valve by the maximum allowable valve position change if: the rate of change of the valve position due to the valve position change based on the proportional and differential parameters is not less than the allowable angular velocity of the control valve; and either: the proportional parameter is not less than the proportional fluctuation constant, or the differential parameter is not less than the differential fluctuation constant; and instructions that cause the one or more processors to update the valve position of the control valve by zero degrees if: the proportional parameter is less than the proportional fluctuation constant; and the differential parameter is less than the differential fluctuation constant.

Other aspects, features, and advantages will become apparent from the following detailed description when taken in conjunction with the accompanying drawings, which are a part of this disclosure and which illustrate, by way of example, principles of the inventions disclosed.

### DESCRIPTION OF FIGURES

The accompanying drawings facilitate an understanding of the various embodiments.

FIG. 1 is a diagrammatic illustration of a mud-gas separator apparatus, according to an exemplary embodiment.

FIG. 2 is a perspective view of a portion of the mud-gas separator apparatus of FIG. 1, according to an exemplary embodiment.

FIG. 3 is a front elevational view of the portion of FIG. 2, according to an exemplary embodiment.

FIG. 4 is a right side elevational view of the portion of FIGS. 2 and 3, according to an exemplary embodiment.

FIG. 5 is a top plan view of the portion of FIGS. 2-4, according to an exemplary embodiment.

FIG. 6 is a perspective view of another portion of the mud-gas separator apparatus of FIG. 1, according to an exemplary embodiment.

FIG. 7 is a perspective view of the portion of FIG. 6 and further illustrates internal components of the mud-gas separator apparatus, according to an exemplary embodiment.

FIG. 8 is a left side elevational view of yet another portion of the mud-gas separator apparatus of FIG. 1 and further illustrates internal components of the mud-gas separator apparatus, according to an exemplary embodiment.

FIG. 9 is a diagrammatic illustration of a mud-gas separator apparatus according to another exemplary embodiment.

FIG. 10 is a flow chart illustration of a method of retrofitting a mud-gas separator apparatus, according to an exemplary embodiment.

FIG. 11 is a diagrammatic illustration of the mud-gas separator apparatus referenced in the method of FIG. 10, according to an exemplary embodiment.

FIG. 12 is a diagrammatic illustration of a mud-gas separator apparatus, according to an exemplary embodiment.

FIG. 13 is a perspective view of components of the mud-gas separator apparatus of FIG. 12, according to an exemplary embodiment.

FIG. 14A is enlarged view of a portion of the components of FIG. 13, according to an exemplary embodiment.

FIG. 14B is a perspective view of some of the components of the mud-gas separator apparatus shown in FIGS. 13 and 14A, according to an exemplary embodiment.

FIG. 15 is a flow chart illustration of a method of controlling a control valve, according to an exemplary embodiment.

FIG. 16 is a diagrammatic illustration of a user interface according to an exemplary experimental embodiment, the user interface including an exemplary experimental embodiment of output generated during a simulation of the execution of the method of FIG. 15.

FIG. 17 is a view similar to that of FIG. 16, but depicting the user interface according to another exemplary experimental embodiment, the user interface including another exemplary experimental embodiment of output generated during a simulation of the execution of the method of FIG. 15.

FIG. 19 is a flow chart illustration of a method of retrofitting a mud-gas separator apparatus, according to an exemplary embodiment.

FIG. 20 is a flow chart illustration of a method of controlling a control valve, according to an exemplary embodiment.

FIG. 21 is a flow chart illustration of a step of the method of FIG. 20, according to an exemplary embodiment.

FIG. 22 is a diagrammatic illustration of the response of an exemplary experimental embodiment of the mud-gas separator apparatus of FIG. 12 to a rapid increase in flow rate, according to an exemplary experimental embodiment.

FIG. 23 is a diagrammatic illustration of the response of an exemplary experimental embodiment of the mud-gas separator apparatus of FIG. 12 to a rapid decrease in flow rate, according to an exemplary experimental embodiment.

FIG. 24 is a diagrammatic illustration of the response of an exemplary experimental embodiment of the mud-gas separator apparatus of FIG. 12 to a disturbance, or temporary spike, in flow rate, according to an exemplary experimental embodiment.

FIG. 25 is a diagrammatic illustration of a node for implementing one or more exemplary embodiments of the present disclosure, according to an exemplary embodiment.

### DETAILED DESCRIPTION

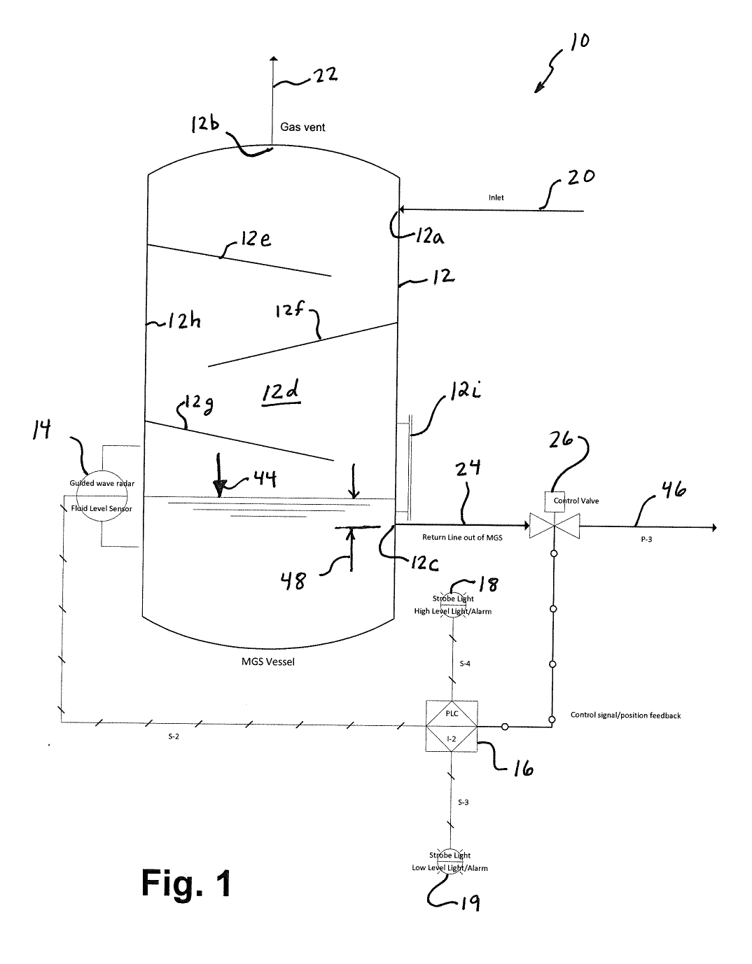

In an exemplary embodiment, as illustrated in FIG. 1, an apparatus is generally referred to by the reference numeral 10 and includes a mud-gas separator vessel 12 and a guided wave level sensor 14 operably coupled thereto. An electronic controller 16 is operably coupled to, and in communication with, the guided wave level sensor 14. A high level alarm 18 is operably coupled to, and in communication with, the electronic controller 16. A low level alarm 19 is operably coupled to, and in communication with, the electronic controller 16. The mud-gas separator vessel 12 includes an inlet 12a at an upper end portion thereof, a gas vent 12b at a top portion thereof, and an outlet 12c at a lower end portion thereof. The mud-gas separator 12 defines an internal region 12d, with which the inlet 12a, the gas vent 12b, and the outlet 12c are in fluid communication. Baffle plates 12e, 12f, and 12g are disposed within the internal region 12a, and are connected to a cylindrical wall 12h of the mud-gas separator vessel 12. In several exemplary embodiments, the baffle plates 12e, 12f, and 12g are omitted from the mud-gas separator vessel 12. A manway 12i is connected to the cylindrical wall 12h, and provides access to the internal region 12. A mud-gas inlet line 20 is in fluid communication with the inlet 12a of the mud-gas separator vessel 12. A gas vent line 22 is in fluid communication with the gas vent 12b of the mud-gas separator vessel 12. A slurry return line 24 is connected to the mud-gas separator vessel 12, and is in fluid communication with the outlet 12c. A control valve 26 is in fluid communication with the slurry return line 24. The electronic controller 16 is connected to, and in communication with, the control valve 26. In several exemplary embodiments, the electronic controller 16 includes one or more processors, a non-transitory computer readable medium operably coupled to the one or more processors, and a plurality of instructions stored on the non-transitory computer readable medium, the instructions being accessible to, and executable by, the one or more processors.

In an exemplary embodiment, the high level alarm 18 is a strobe light high level light/alarm. In an exemplary embodiment, the low level alarm 19 is a strobe light low level light/alarm.

In several exemplary embodiments, the guided wave level sensor 14 is, includes, or is part of, a Magnetrol® Eclipse® Model 706 high performance guided wave radar level transmitter, which is available from Magnetrol International, Incorporated, Downers Grove, Ill. USA.

In several exemplary embodiments, the electronic controller 16 is, includes, or is part of, a programmable logic controller (PLC). In several exemplary embodiments, the electronic controller 16 is, includes, or is part of, a programmable logic controller from the CP1 family of compact machine controllers, which are available from the Omron Corporation, Tokyo, Japan.

In several exemplary embodiments, the control valve 26 is, includes, or is part of, a Fisher® Vee-Ball™ V150, V1200, or V300 rotary control valve, each of which is available from Emerson Process Management, Marshalltown, Iowa USA.

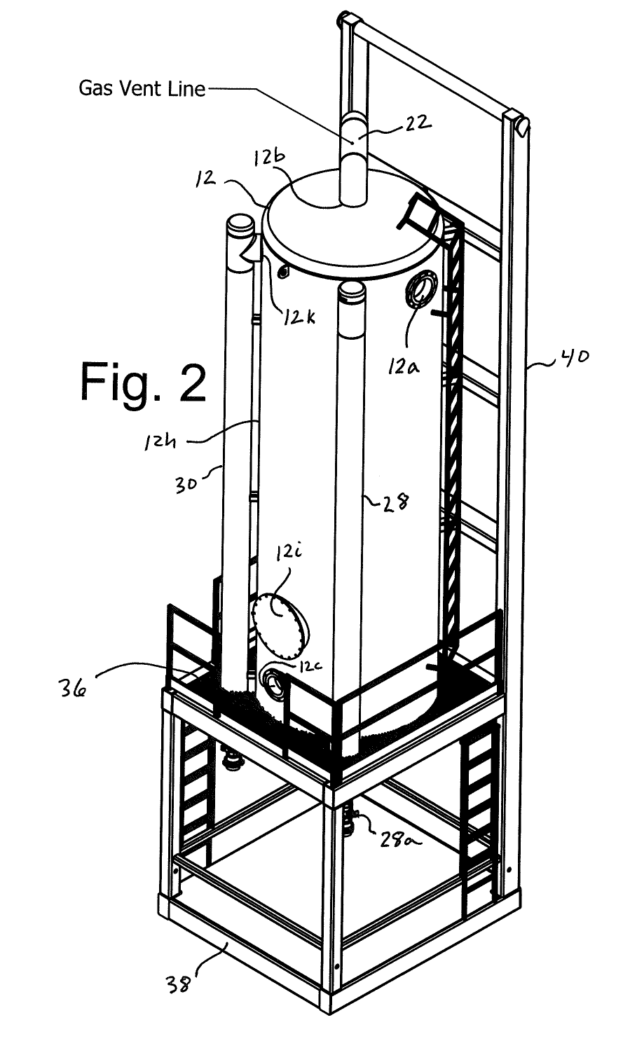

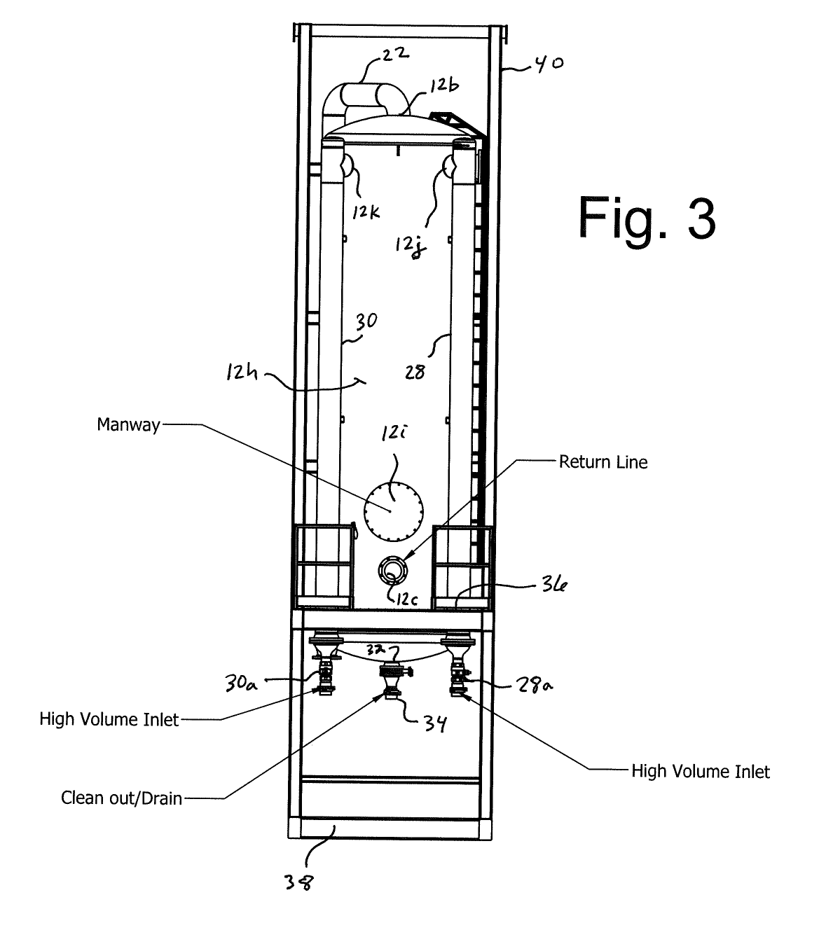

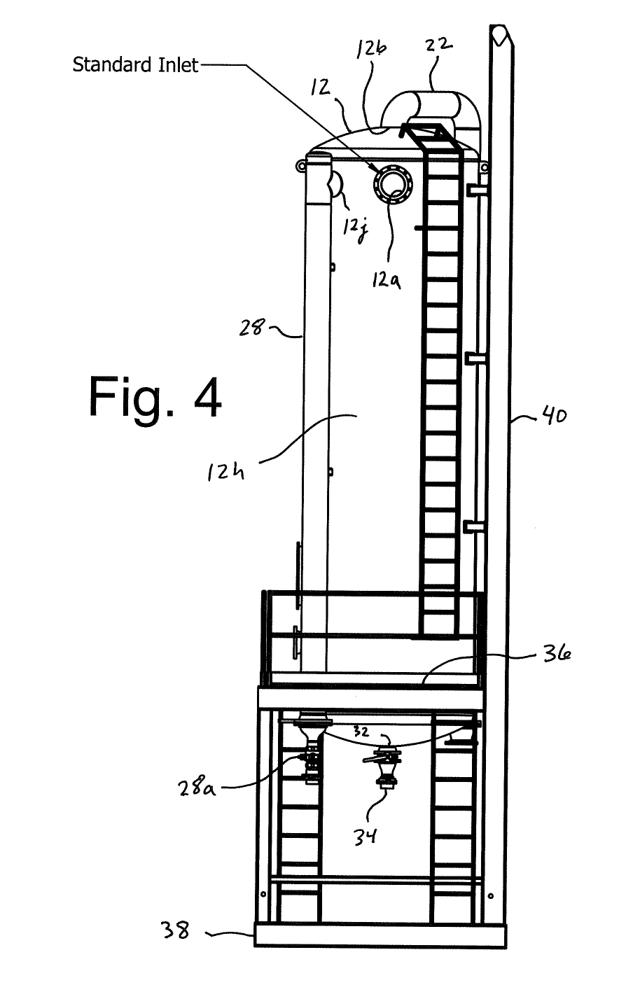

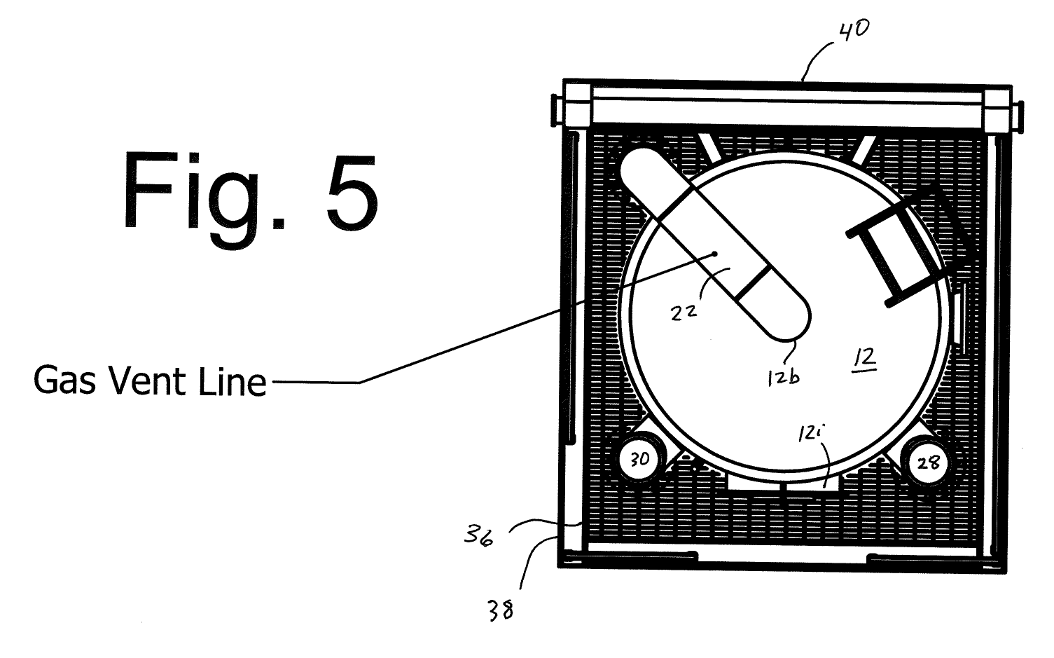

In an exemplary embodiment, as illustrated in FIGS. 2-5 with continuing reference to FIG. 1, the mud-gas separator vessel 12 further includes circumferentially-spaced high volume inlets 12j and 12k at the upper end portion thereof. Parallel-spaced high volume inlet lines 28 and 30 are connected to the mud-gas separator vessel 12, extending vertically from below the mud-gas separator vessel 12, along the cylindrical wall 12h thereof, and to the upper end portion thereof. The high volume inlet lines 28 and 30 are in fluid communication with the high volume inlets 12j and 12k, respectively. The high volume inlet lines 28 and 30 include valves 28a and 30a, respectively, at lower end portions thereof. A drain outlet 32 is formed through the bottom of the mud-gas separator vessel 12. A clean-out line 34 is connected to the bottom of the mud-gas separator vessel 12 and is in fluid communication with the drain outlet 32. The mud-gas separator vessel 12 is mounted on a platform 36, which is supported by a base frame 38. The high volume inlet lines 28 and 30 extend through the platform 36. At least the bottom of the mud-gas separator vessel 12 and the clean-out line 34 extend below the platform 36. A vertically-extending frame 40 extends upward from the base frame 38 and alongside the mud-gas separator vessel 12. The vertically-extending frame 40 is connected to at least the cylindrical wall 12h of the mud-gas separator vessel 12.

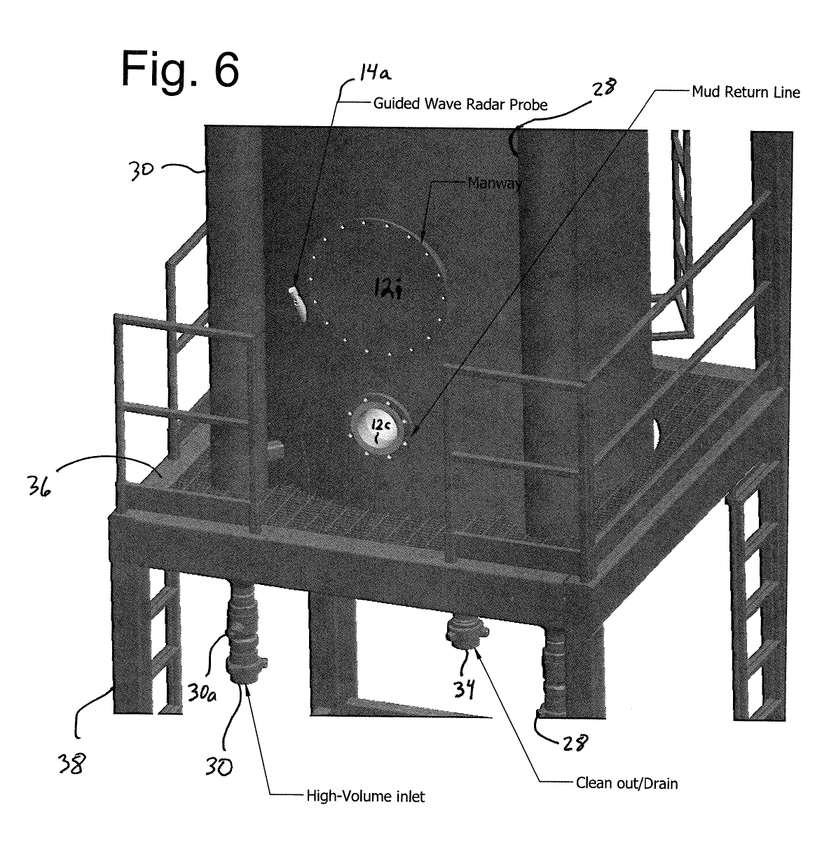

In an exemplary embodiment, as illustrated in FIG. 6 with continuing reference to FIGS. 1-5, the guided wave level sensor 14 includes a guided wave radar probe 14a. The guided wave radar probe 14a extends through the cylindrical wall 12h at a location proximate the manway 12i, and into the internal region 12d.

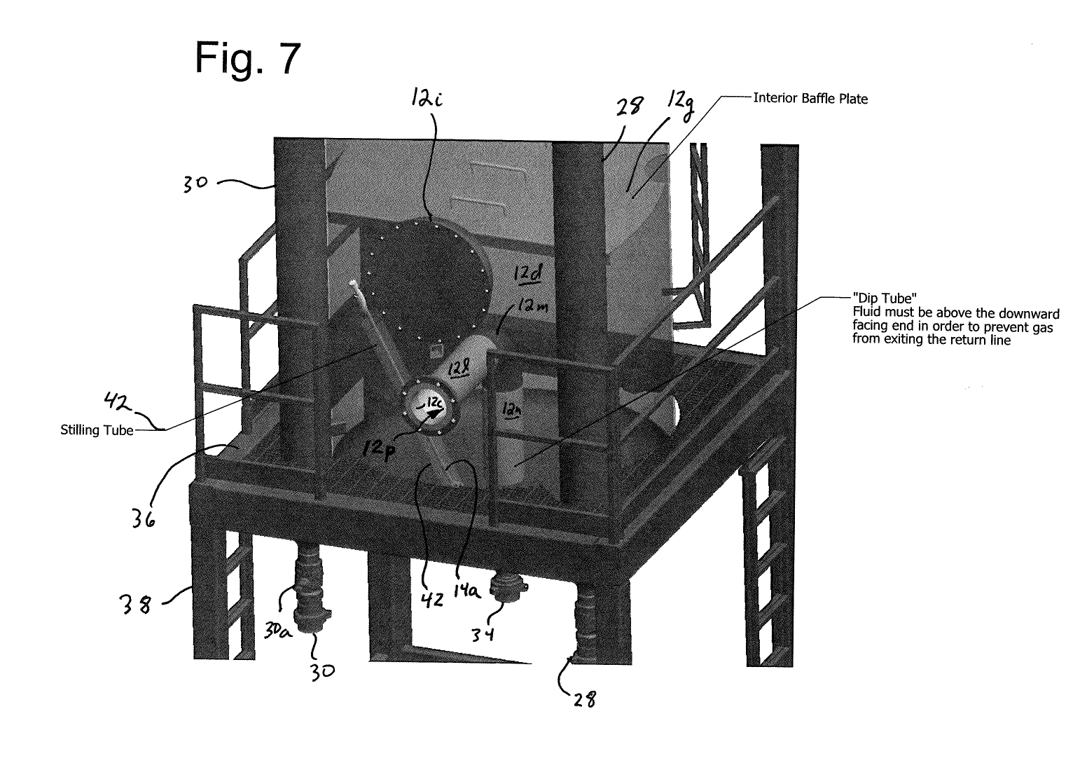

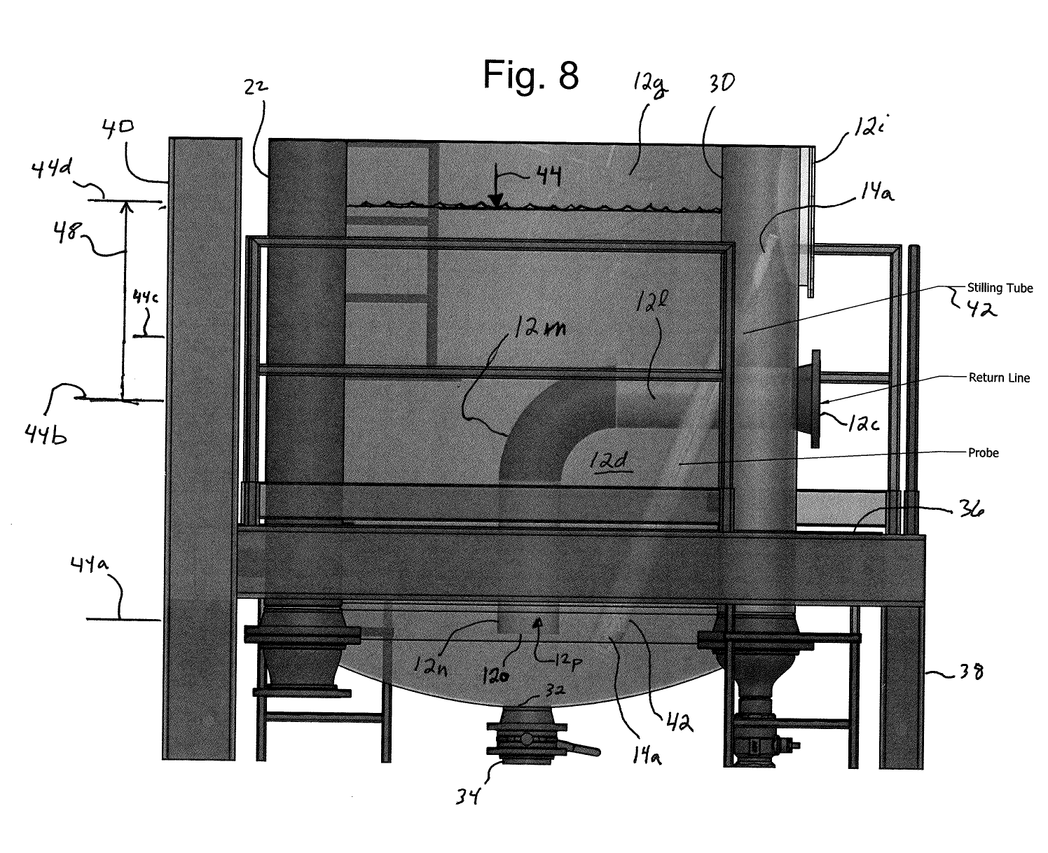

In an exemplary embodiment, as illustrated in FIGS. 7 and 8 with continuing reference to FIGS. 1-6, the outlet 12c of the mud-gas separator vessel 12 includes a horizontally-extending segment 121 extending into the internal region 12d, a joint 12m disposed in the internal region 12d and connected to the horizontally-extending segment 121, and a vertically-extending segment 12n disposed in the internal region 12d and extending downwards from the joint 12m. In an exemplary embodiment, the joint 12m is a 90-degree, or elbow, fitting. A downward-facing end 12o of the vertically-extending segment 12n is located near the drain outlet 32. A fluid passage 12p is defined by at least the end 12o, the vertically-extending segment 12n, the joint 12m, and the horizontally-extending segment 121. The internal region 12d is in fluid communication with the slurry return line 24 via at least the fluid passage 12p. In several exemplary embodiments, the horizontally-extending segment 121, the joint 12m, and the vertically-extending segment 12n may be characterized as a “dip tube.”

A stilling tube 42 extends within the internal region 12d, from a location near the manway 12i to a location near the end 12o of the vertically-extending segment 12n. The guided wave radar probe 14a extends within the stilling tube 42.

In operation, in an exemplary embodiment, a multiphase flow travels through the mud-gas inlet line 20, and into the internal region 12d of the mud-gas separator vessel 12 via the inlet 12a and/or one or both of the high volume inlet lines 28 and 30. The multiphase flow traveling through the mud-gas inlet line 20 includes solid, liquid, and gas materials. In several exemplary embodiments, the multiphase flow traveling through the mud-gas inlet line 20 includes drilling fluid (or drilling mud) having free gas therewithin; this drilling mud may be used in oil and gas exploration and production operations. After entering the internal region 12d, the multiphase flow impinges the baffles 12e, 12f, and 12g, separating the gas materials from the solid and liquid materials in the multiphase flow. Within the internal region 12d, gravitational forces also cause the gas materials to separate from the solid and liquid materials in the multiphase flow. The separated gas materials rise upwards and flow out of the mud-gas separator vessel 12 and into the gas vent line 22 via the gas vent 12b. The remaining solid and liquid materials (hereinafter the “slurry”) collect in the lower end portion of the mud-gas separator vessel 12, defining a fluid level 44 within the internal region 12d. Over time, the fluid level 44 rises, and the slurry rises to the end 12o and into the portion of the fluid passage 12p defined by the vertically-extending segment 12n. The fluid level 44 continues to rise and, when the fluid level 44 reaches a predetermined level, at least a portion of the slurry is discharged from the mud-gas separator vessel 12, flowing from the mud-gas separator vessel 12 and into the slurry return line 24 via the outlet 12c. The slurry flows through the slurry return line 24, the control valve 26, and additional flow line(s) 46, which are part of the slurry return line 24.

During operation, the fluid level 44 is vertically higher than the vertical location of the end 12o to prevent any gas materials from exiting the mud-gas separator vessel 12 via the flow passage 12p and the outlet 12c, that is, to prevent “vent gas carry under.” As a result, any risk of fire due to the gas materials is reduced. The slurry within the internal region 12d provides a liquid seal that prevents vent gas carry under. During operation, to prevent any gas materials from exiting the mud-gas separator vessel 12 via the flow passage 12p and the outlet 12c, the guided wave level sensor 14 measures the fluid level 44 and communicates data associated with the measurement to the electronic controller 16. The electronic controller 16, in turn, automatically controls the control valve 26 based on the measurement data received from the guided wave level sensor 14. The automatic control of the control valve 26 controls the discharge of the slurry out of the mud-gas separator vessel 12 via the slurry return line 24. In several exemplary embodiments, based on the measurement data received from the guided wave level sensor 14, the electronic controller 16: further opens the control valve 26, allowing more slurry to flow through the slurry return line 24 and thus reducing the fluid level 44; further closes the control valve 26, reducing the amount of slurry that flows through the slurry return line 24 and thus increasing the fluid level 44; or maintains the current valve position of the control valve 26, the current valve position of the control valve 26 being at a fully open valve position, a fully closed valve position, or a partially open valve position. As a result, the fluid level 44 can be automatically maintained within a predetermined range within the mud-gas separator vessel 12 to prevent vent gas carry under therefrom; the automatic control of the control valve 26 by the electronic controller 16 automatically controls the discharge of the slurry out of the mud-gas separator vessel 12 and thus automatically maintains the fluid level within the predetermined range.

During operation, if the controller 16 determines that the fluid level 44 is too high (i.e., is at, or exceeds, a predetermined high level), the controller 16 activates the high level alarm 18. During operation, if the controller 16 determines that the fluid level 44 is too low (i.e., is at, or is below, another predetermined low level), the controller 16 activates the low level alarm 19.

In several exemplary embodiments, the combination of the guided wave level sensor 14, the electronic controller 16, and the control valve 26 provides intelligent system control of slurry discharge from the mud-gas separator vessel 12, thereby actively preventing vent gas carry under. In several exemplary embodiments, the apparatus 10 maintains the liquid seal provided by the slurry, thereby preventing vent gas carry under.

In several exemplary embodiments, due to the intelligent system control, or active control, of the fluid level 44 by the combination of the guided wave level sensor 14, the electronic controller 16, and the control valve 26, the need to include a U-tube in the slurry return line 24 is eliminated, thereby greatly reducing the footprint, and/or the volume, of the apparatus 10 (that is, how much ground space the apparatus 10 takes up, and/or how much volumetric space the apparatus 10 takes up). As a result, the apparatus 10, or one or more components thereof, are easier to transport and install.

In several exemplary embodiments, a vertical distance between the fluid level 44 and the slurry return line 24, or between the fluid level 44 and the downwardly-facing end 12o (or another portion of the segment 121, the joint 12m, or the segment 12n), must be high enough to reduce the risk of vent gas carry under. As shown in FIG. 1, this vertical distance is referred to as mud leg 48. In several exemplary embodiments, due to the intelligent system control, or active control, of the fluid level 44 in the apparatus 10, the need to unnecessarily increase the mud leg 48 is eliminated, thereby allowing the mud-gas separator vessel 12 to operate more efficiently and separate more multiphase flow. In several exemplary embodiments, the mud leg 48 may be reduced from, for example, 6 feet to 4 feet.

In several exemplary embodiments, instead of, or in addition to one or both of the alarms 18 and 19, the electronic controller 16 may include a plurality of alarms. In several exemplary embodiments, instead of, or in addition to one or both of the alarms 18 and 19, the apparatus 10 may include one or more other alarms.

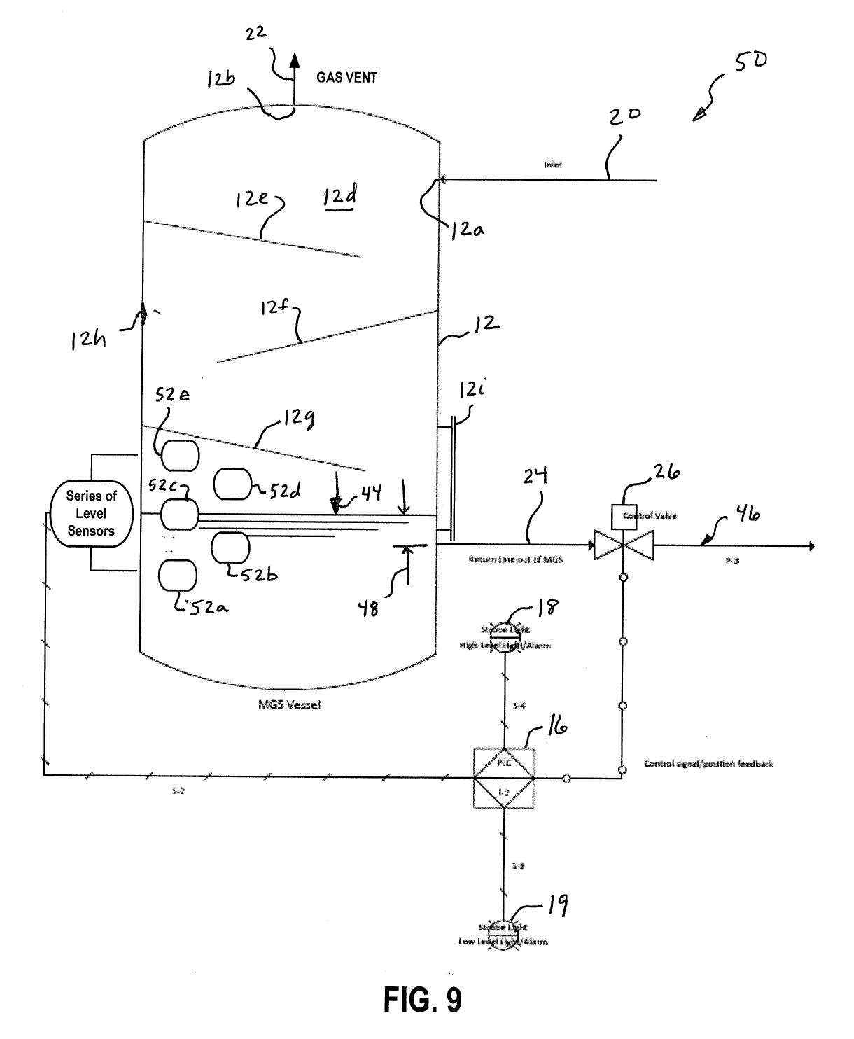

In an exemplary embodiment, as illustrated in FIG. 9 with continuing reference to FIGS. 1-8, an apparatus is generally referred to by the reference numeral 50 and includes the great majority of the components of the apparatus 10, which components are given the same reference numerals. In the apparatus 50 illustrated in FIG. 9, the guided wave level sensor 14 is omitted, and the apparatus 50 instead includes a series of level sensors 52a, 52b, 52c, 52d, and 52e, all of which are operably coupled to the mud-gas separator vessel 12. In several exemplary embodiments, at least one of the level sensors 52a, 52b, 52c, 52d, and 52e includes a vibrating fork level switch. In several exemplary embodiments, at least one of the level sensors 52a, 52b, 52c, 52d, and 52e is, includes, or is part of, a Rosemount® 2130 Enhanced Vibrating Fork Liquid Level Switch, which is available from Emerson Process Management Rosemount Inc., Chanhassen, Minn. USA. In several exemplary embodiments, the operation of the apparatus 50 is substantially identical to the operation of the apparatus 10, except that, instead of the guided wave level sensor 14, one or more of the level sensors 52a, 52b, 52c, 52d, and 52e measure the fluid level 44 and communicate the measurement(s) to the electronic controller 16. Therefore, the operation of the apparatus 50 will not be described in further detail.

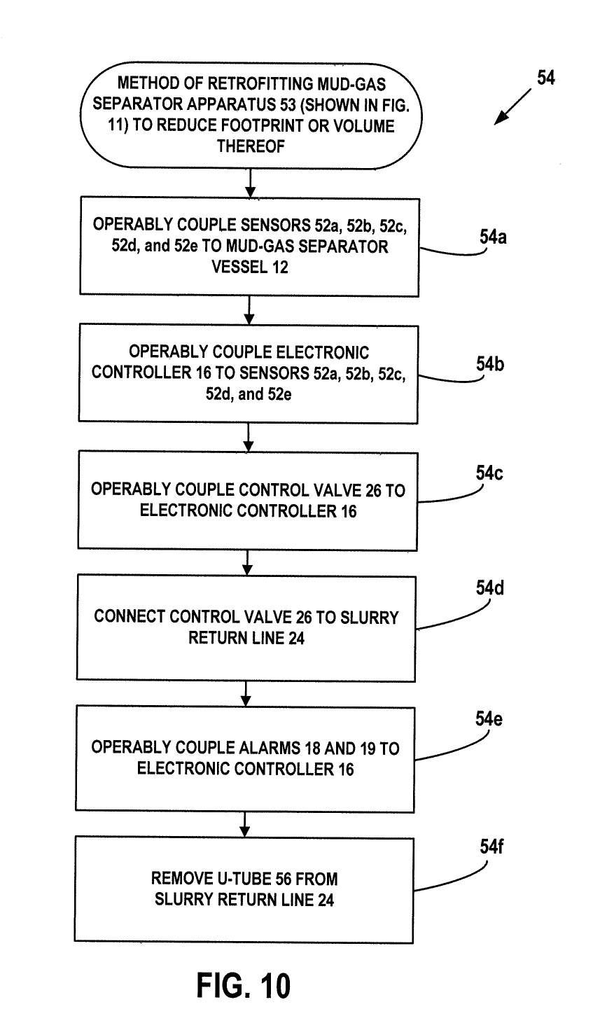

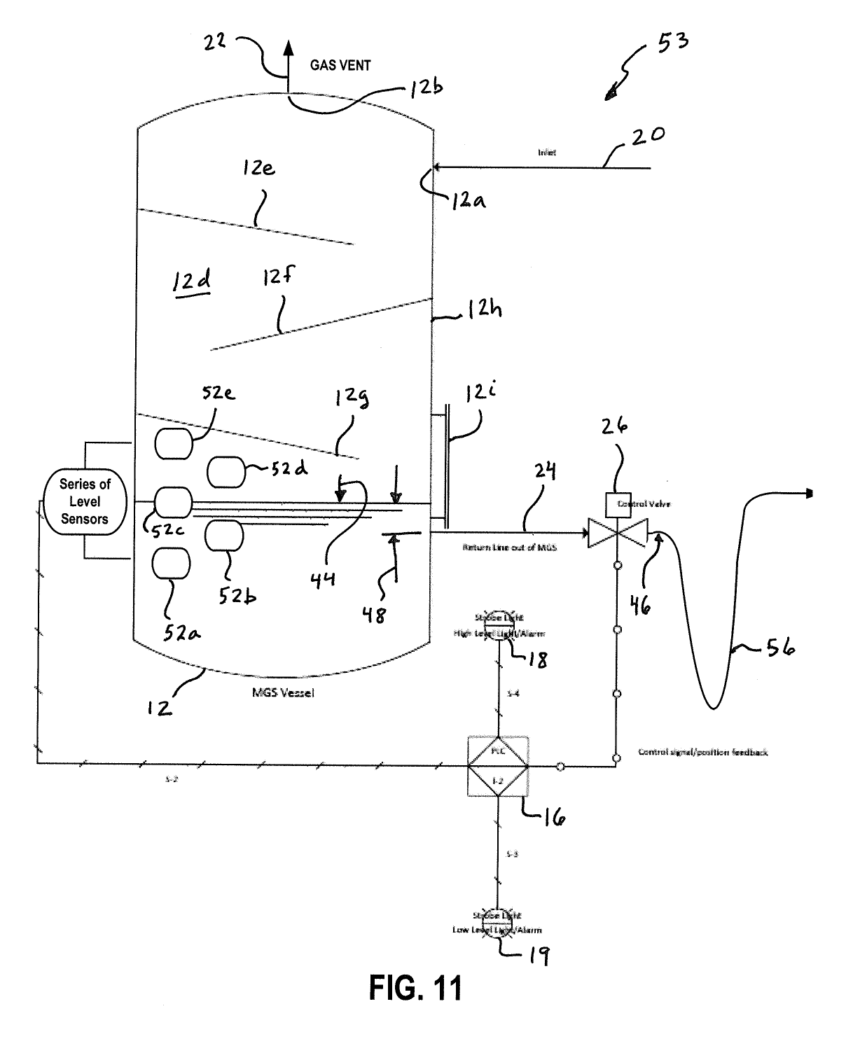

In an exemplary embodiment, as illustrated in FIGS. 10 and 11 with continuing reference to FIGS. 1-9, a method of retrofitting a mud-gas separator apparatus 53 (shown in FIG. 11) to reduce the footprint or volume thereof is generally referred to by the reference numeral 54. As shown in FIG. 11, the mud-gas separator apparatus 53 is identical to the mud-gas separator apparatus 50, except that the additional flow line(s) 46 of the slurry return line 24 of the mud-gas separator apparatus 53 include a U-tube 56. The U-tube 56 ensures a liquid seal to prevent vent gas carry under from the mud-gas separator vessel 12, but also significantly increases the footprint or volume of the mud-gas separator apparatus 53. The method 54 includes: at step 54a operably coupling the sensors 52a, 52b, 52c, 52d, and 52e to the mud-gas separator vessel 12; at step 54b operably coupling the electronic controller 16 to the sensors 52a, 52b, 52c, 52d, and 52e; at step 54c operably coupling the control valve 26 to the electronic controller 16; at step 54e operably coupling the alarms 18 and 19 to the electronic controller 16; and at step 54f removing the U-tube 56 from the slurry return line 24, thereby reducing the footprint or volume of the mud-gas separator apparatus 53. The U-tube 56 is removed at the step 54f because the U-tube 56 is no longer needed to ensure that a liquid seal is maintained to prevent vent gas carry under from the mud-gas separator vessel 12. The U-tube 56 is no longer needed because of the active control of the liquid level 44 provided by the operation of the combination of the sensors 52a, 52b, 52c, 52d, and 52e, the electronic controller 16, and the control valve 26. In several exemplary embodiments, the guided wave level sensor 14 may be operably coupled to the mud-gas separator vessel 12 at the step 54a, and the electronic controller 16 may be operably coupled to the guided wave level sensor 14 at the step 54b.

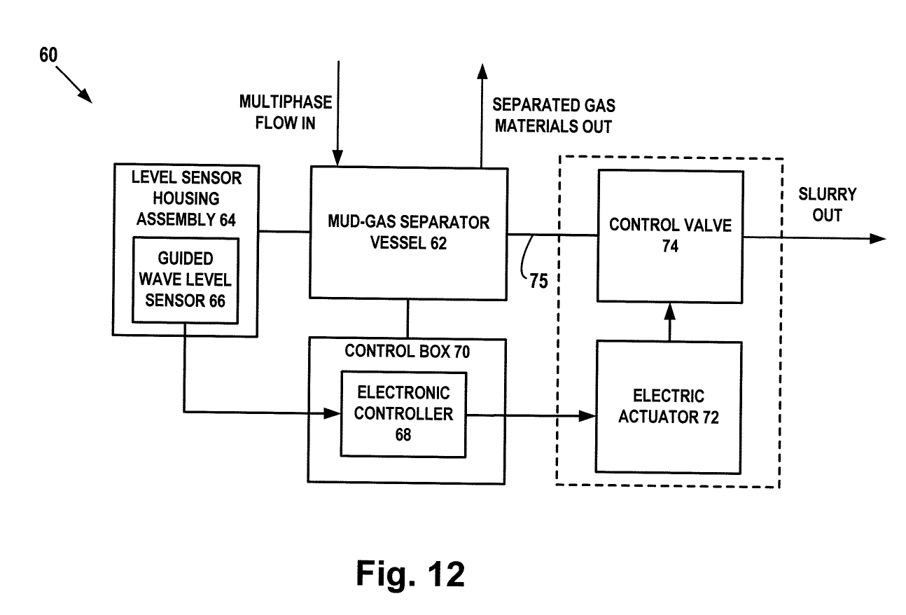

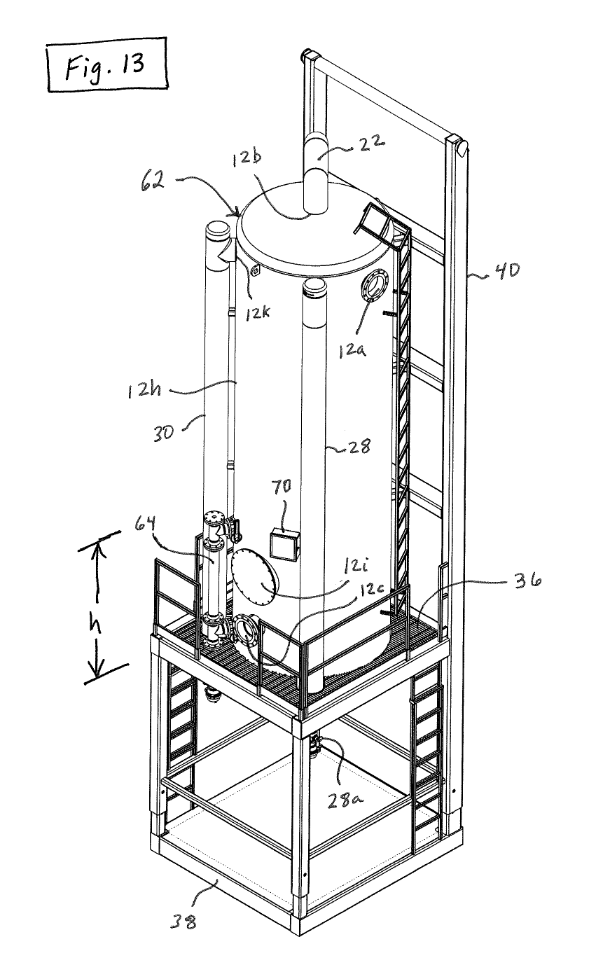

In an exemplary embodiment, as illustrated in FIG. 12 with continuing reference to FIGS. 1-11, an apparatus is generally referred to by the reference numeral 60 and includes a mud-gas separator vessel 62 and a level sensor housing assembly 64 connected thereto. At least a portion of a guided wave level sensor 66 is housed within the level sensor housing assembly 64. An electronic controller 68 is operably coupled to, and in communication with, the guided wave level sensor 66. A control box 70 is connected to the mud-gas separator vessel 62. At least a portion of the electronic controller 68 is housed within the control box 70. An electric actuator 72 is operably coupled to, and in communication with, the electronic controller 68. A control valve 74 is operably coupled to the electric actuator 72. In several exemplary embodiments, the electric actuator 72 is part of the control valve 74, and the control valve 74 is in communication with the electronic controller 68 via the electric actuator 72. As indicated in FIG. 12 and as will be described in further detail below, in several exemplary embodiments, a multiphase flow is adapted to flow into the mud-gas separator vessel 62, gas materials are adapted to separate from solid and liquid materials within the mud-gas separator vessel 62, the separated gas materials are adapted to flow out of the mud-gas separator vessel 62 via a gas vent, and the solid and liquid materials are adapted to flow out of the mud-gas separator vessel 62 and through the control valve 74. In an exemplary embodiment, the control valve 74 is connected to a slurry return line 75, and the control valve 74 is in fluid communication with the mud-gas separator vessel 62 via at least the slurry return line 75. In an exemplary embodiment, the slurry return line 75 is part of the mud-gas separator vessel 62. As will be described in further detail below, in several exemplary embodiments, the control valve 74 is automatically controlled by the respective operations of the guided wave level sensor 66, the electronic controller 68, and the electric actuator 72.

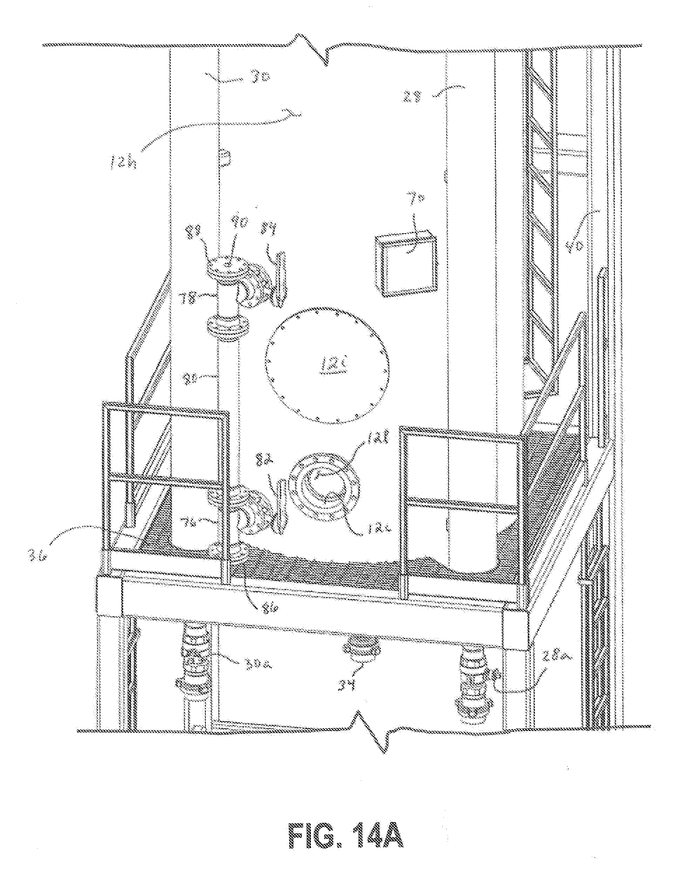

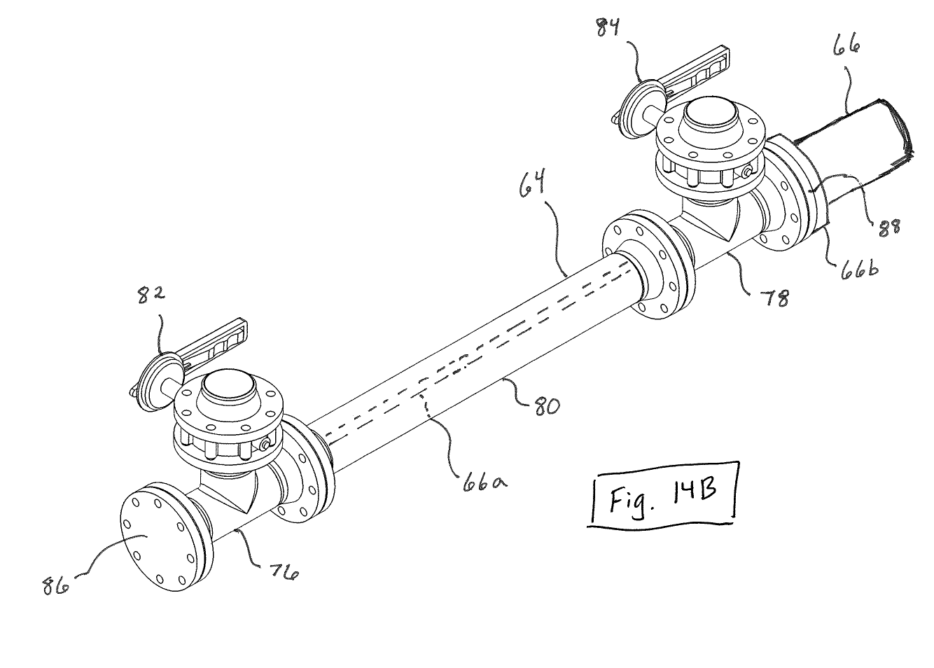

In an exemplary embodiment, as illustrated in FIGS. 13, 14A, and 14B with continuing reference to FIGS. 1-12, the mud-gas separator 62 includes all of the components of the mud-gas separator 12 as shown in FIGS. 2-8, which components are given the same reference numerals. Moreover, the apparatus 60 includes several other components of the apparatus 10 as shown in FIGS. 1-8, which components are given the same reference numerals. However, in contrast to the apparatus 10 as shown in FIGS. 7 and 8, the apparatus 60 does not include the guided wave level sensor 14 and thus the guided wave radar probe 14a thereof does not extend through the cylindrical wall 12h of the mud-gas separator vessel 62. Further, the apparatus 60 does not include the stilling tube 42 and thus the stilling tube 42 does not extend within the internal region 12d. The guided wave level sensor 14 and the stilling tube 42 are omitted from the apparatus 60 in favor of the guided wave level sensor 66 and the level sensor housing assembly 64, respectively.

In the mud-gas separator vessel 62 shown in FIGS. 13, 14A, and 14B, the level sensor housing assembly 64 is connected to the exterior of the cylindrical wall 12h of the mud-gas separator 62. The control box 70 is also connected to the exterior of the cylindrical wall 12h of the mud-gas separator 62 at a location proximate the manway 12i of the mud-gas separator 62. Although not shown in FIGS. 13 and 14A, the control valve 74 is in fluid communication with the outlet 12c of the mud-gas separator vessel 62.

As shown more clearly in FIGS. 14A and 14B, the level sensor housing assembly 64 includes a lower t-shaped fitting 76 and an upper t-shaped fitting 78 vertically spaced therefrom. A tubular member 80 is connected to, and extends vertically between, the fittings 76 and 78. The tubular member 80 is spaced from the exterior of the cylindrical wall 12h. The tubular member 80 is in fluid communication with each of the fittings 76 and 78. Isolation valves 82 and 84 are connected to, and in fluid communication with, the fittings 76 and 78, respectively. The isolation valves 82 and 84 are connected to the cylindrical wall 12h of the mud-gas separator vessel 62, and are in fluid communication with the internal region 12d of the mud-gas separator vessel 62 via respective openings (not shown) formed through the cylindrical wall 12h. The isolation valve 82 is proximate the outlet 12c, and the isolation valve 84 is proximate the manway 12i. In an exemplary embodiment, as show in FIGS. 13 and 14A, the isolation valve 82 is vertically positioned below the outlet 12c and thus below the horizontally-extending segment 121. The tubular member 80 is in fluid communication with the internal region 12d of the mud-gas separator vessel 62 via the isolation valves 82 and 84 and the fittings 76 and 78. The fitting 76 includes a solid cap 86 at the base thereof; in several exemplary embodiments, the cap 86 rests against, or is at least proximate, the platform 36. The fitting 78 includes a cap 88 at the top thereof. An opening, or insertion port 90 (FIG. 14A), is formed through the cap 88; a rod-shaped probe 66a (FIG. 14B) of the guided wave level sensor 66 is adapted to extend through the insertion port 90.

In several exemplary embodiments, instead of being t-shaped, the fittings 76 and 78 may be either y-shaped or cross-shaped, or may have other shapes. In several exemplary embodiments, the tubular member 80 may be integrally formed with one or both of the fittings 76 and 78. In several exemplary embodiments, the isolation valves 82 and 84 may be integrally formed with the fittings 76 and 78, respectively.

In an exemplary embodiment, the guided wave level sensor 66 is a LevelFlex FMP51 rod-type level sensor, which is available from Endress+Hauser Inc., Greenwood, Ind. USA. In an exemplary embodiment, the guided wave level sensor 66 is connected to the cap 88 of the fitting 78, and the rod-shaped probe 66a (FIG. 14B) of the guided wave level sensor 66 extends through the insertion port 90, through the fitting 78, and at least within the tubular member 80. In several exemplary embodiments, the rod-shaped probe 66a of the guided wave level sensor 66 extends through the fitting 78, through the tubular member 80, and within the fitting 76. In several exemplary embodiments, the guided wave level sensor 66 is connected to the cap 88 via a flange connection 66b (FIG. 14B).

In an exemplary embodiment, the electronic controller 68 is, includes, or is part of, a CompactRIO embedded system, which is available from National Instruments Corporation, Austin, Tex. USA. In an exemplary embodiment, the electronic controller 68 is, includes, or is part of, a NI Single-Board RIO embedded system, which is available from National Instruments Corporation, Austin, Tex. USA.

In an exemplary embodiment, the electric actuator 72 is a Bettis EM-500 Series actuator, which is available from Bettis Electric, Mansfield, Ohio USA. In several exemplary embodiments, the actuator 72 is not an electric actuator and instead is another type of actuator.

In an exemplary embodiment, the control valve 74 is a rotary control valve. In an exemplary embodiment, the control valve 74 is a Fisher® Vee-Ball™ V150 rotary control valve, which is available from Emerson Process Management, Marshalltown, Iowa USA. In several exemplary embodiments, the electric actuator 72 is part of the control valve 74, and/or the components together may be referred to as a control valve that is operably coupled to, and in communication with, the electronic controller 68.

In operation, in an exemplary embodiment, a multiphase flow travels into the internal region 12d of the mud-gas separator vessel 62 via the inlet 12a and/or one or both of the high volume inlet lines 28 and 30; the multiphase flow includes solid, liquid, and gas materials. In several exemplary embodiments, the multiphase flow traveling into the internal region 12d includes drilling fluid (or drilling mud) having free gas therewithin; this drilling mud may be used in oil and gas exploration and production operations. After entering the internal region 12d, the multiphase flow impinges one or more baffles, such as the baffles 12e, 12f, and 12g, separating the gas materials from the solid and liquid materials in the multiphase flow. Within the internal region 12d, gravitational forces also cause the gas materials to separate from the solid and liquid materials in the multiphase flow. In several exemplary embodiments, the baffle plates 12e, 12f, and 12g are omitted from the mud-gas separator vessel 12, and the separation of the gas materials from the solid and liquid materials is primarily caused by gravitational forces.

The separated gas materials rise upwards and flow out of the mud-gas separator vessel 62 and into the gas vent line 22 via the gas vent 12b. The remaining solid and liquid materials (hereinafter the “slurry”) collect in the lower end portion of the mud-gas separator vessel 62, defining the fluid level 44 (shown in FIGS. 1 and 8) within the internal region 12d. Over time, the fluid level 44 rises, and the slurry rises to the end 12o and into the portion of the fluid passage 12p defined by the vertically-extending segment 12n. The fluid level 44 continues to rise and, when the fluid level 44 reaches a predetermined level, at least a portion of the slurry is discharged from the mud-gas separator vessel 12, flowing out of the mud-gas separator vessel 62 via the outlet 12c. The slurry subsequently flows through the control valve 74 and additional flow line(s) downstream thereof.

During operation, the fluid level 44 is vertically higher than the vertical location of the end 12o to prevent any gas materials from exiting the mud-gas separator vessel 62 via the flow passage 12p and the outlet 12c, that is, to prevent “vent gas carry under.” As a result, any risk of fire due to the gas materials is reduced. The slurry within the internal region 12d provides a liquid seal that prevents vent gas carry under. During operation, to prevent any gas materials from exiting the mud-gas separator vessel 62 via the flow passage 12p and the outlet 12c, the guided wave level sensor 66 measures the fluid level 44 and communicates data associated with the measurement to the electronic controller 68. The electronic controller 68 reads the data and, in turn, automatically controls the electric actuator 72, which opens, further opens, or further closes the control valve 74 based on the measurement data received from the guided wave level sensor 66; thus, the electronic controller 68 automatically controls the control valve 74. The automatic control of the control valve 74 controls the discharge of the slurry out of the mud-gas separator vessel 62. In several exemplary embodiments, based on the measurement data received from the guided wave level sensor 66, the electronic controller 68: opens or further opens the control valve 74, allowing more slurry to flow out of the internal region 12d and thus reducing the fluid level 44; further closes the control valve 74, reducing the amount of slurry that flows out of the internal region 12d and thus increasing the fluid level 44; or maintains the current valve position of the control valve 74, the current valve position of the control valve 74 being at a fully open valve position, a fully closed valve position, or a partially open valve position. As a result, the fluid level 44 can be automatically maintained within a predetermined range within the mud-gas separator vessel 62 to prevent vent gas carry under therefrom; the automatic control of the control valve 74 by the electronic controller 68 automatically controls the discharge of the slurry out of the mud-gas separator vessel 62 and thus automatically maintains the fluid level within the predetermined range. In several exemplary embodiments, the predetermined range is based on a desired value for the fluid level 44, plus an acceptable increase thereabove and minus an acceptable decrease therebelow; thus, the predetermined range extends from a first level, which equals the desired value for the fluid level 44 minus the acceptable decrease therebelow, to a second level, which equals the desired value for the fluid level 44 plus the acceptable increase thereabove.

In several exemplary embodiments, the combination of the guided wave level sensor 66, the electronic controller 68, the electric actuator 72, and the control valve 74 provides intelligent system control of slurry discharge from the mud-gas separator vessel 62, thereby actively controlling the fluid level 44 and actively preventing vent gas carry under. In several exemplary embodiments, the apparatus 60 maintains the liquid seal provided by the slurry, thereby preventing vent gas carry under.

In several exemplary embodiments, due to the intelligent system control, or active control, of the fluid level 44 by the combination of the guided wave level sensor 66, the electronic controller 68, the electric actuator 72, and the control valve 74, the need to include a U-tube downstream of the control valve 74 is eliminated, thereby greatly reducing the footprint, and/or the volume, of the apparatus 60 (that is, how much ground space the apparatus 60 takes up, and/or how much volumetric space the apparatus 60 takes up). As a result, the apparatus 60, or one or more components thereof, are easier to transport and install.

In several exemplary embodiments, the electronic controller 68 may include one or more alarms, and during operation may activate the one or more alarms when the fluid level 44 is too high (i.e., is at, or exceeds, a predetermined high level). In several exemplary embodiments, during operation, the electronic controller 68 may activate one or more alarms when the fluid level 44 is too low (i.e., is at, or is below, another predetermined low level). Instead of, or in addition to, activating one or more alarms, the electronic controller 68 may take other action(s) when the fluid level 44 is too high or too low.

In several exemplary embodiments, a vertical distance between the fluid level 44 and the outlet 12c, or between the fluid level 44 and the downwardly-facing end 12o (or another portion of the segment 121, the joint 12m, or the segment 12n), must be high enough to reduce the risk of vent gas carry under. As shown in FIGS. 1 and 8, this vertical distance is referred to as the mud leg 48. In several exemplary embodiments, due to the intelligent system control, or active control, of the fluid level 44 in the apparatus 60, the need to unnecessarily increase the mud leg 48 is eliminated, thereby allowing the mud-gas separator vessel 62 to operate more efficiently and separate more multiphase flow, that is, separate gas materials in the multiphase flow from the remaining solid and liquid materials in the multiphase flow.

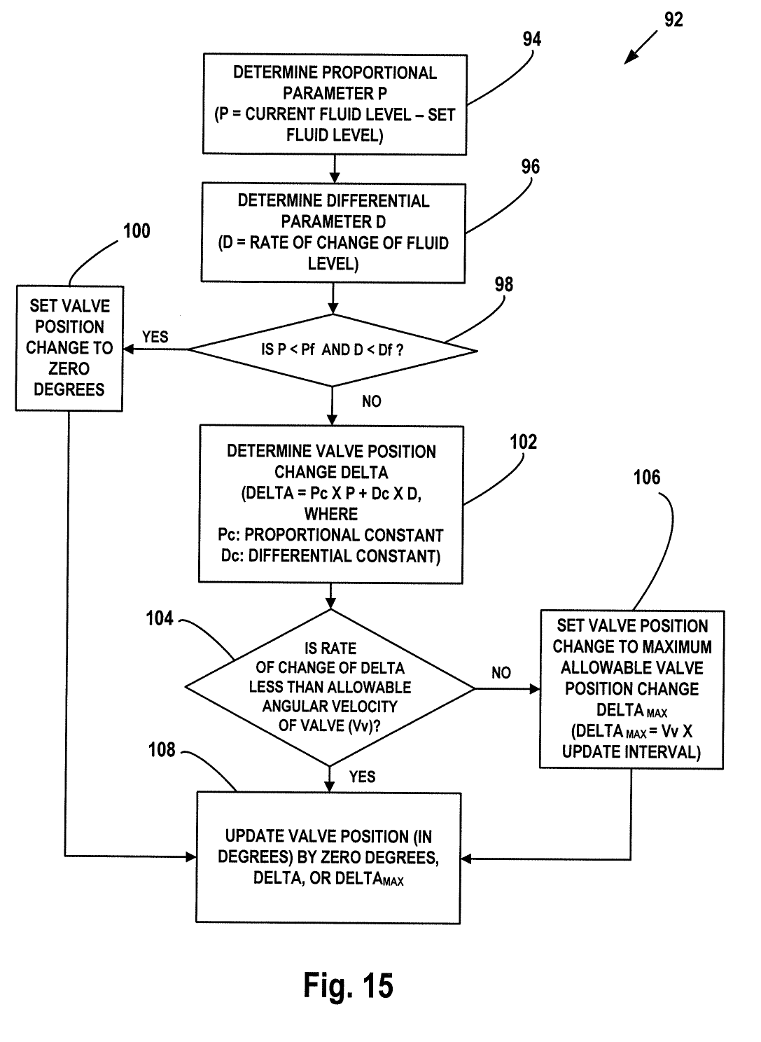

In an exemplary embodiment, as illustrated in FIG. 15 with continuing reference to FIGS. 1-14B, a method of controlling the control valve 74, to actively control the fluid level 44 within the internal region 12d (or the mud leg 48 which is based on the fluid level 44), is generally referred to by the reference numeral 92. In several exemplary embodiments, the method 92 is repeatedly executed during the above-described operation of the apparatus 60.

In an exemplary embodiment, the method 92 is executed by the operation of the electronic controller 68, which is, includes, or is part of, a proportional derivative (PD) controller, as well as by the above-described operation of the guided wave level sensor 66, the electric actuator 72, and the control valve 74.

In the method 92, a proportional parameter P is determined at step 94. At the step 94, in an exemplary embodiment, the proportional parameter P is equal to the current fluid level 44, as currently measured by the guided wave level sensor 66, minus a set fluid level, which is the desired value for the fluid level 44 (P=Current Fluid Level−Set Fluid Level). At the step 94, the electronic controller 68 reads data associated with the fluid level 44 from the guided wave level sensor 66, which transmits the data to the electronic controller 68. In several exemplary embodiments, the electronic controller 68 reads the data associated with the fluid level 44 from one or more measurements by the guided wave level sensor 66 taken at the end of an update time interval of, for example, every 1 second, 5 seconds, or 10 seconds.

At step 96, a differential parameter D is determined. At the step 96, in an exemplary embodiment, the differential parameter D is equal to the rate of change of the fluid level 44. At step 98, it is determined whether the proportional parameter P is less than a proportional fluctuation constant Pf, and whether the differential parameter D is less than a differential fluctuation constant Df. If it is determined at the step 98 that the proportional parameter P is less than the proportional fluctuation constant Pf, and that the differential parameter D is less than the differential fluctuation constant Df, then at step 100 the change in the valve position of the control valve 74 is set to zero (0) degrees, that is, the valve position of the control valve 74 is not to be changed. If it is determined at the step 98 that the proportional parameter P is not less than the proportional fluctuation constant Pf, and/or that the differential parameter D is not less than the differential fluctuation constant Df, then at step 102 a valve position change Delta is determined. At the step 102, a valve position change Delta is equal to the product of a proportional constant Pc and the proportional parameter P, plus the product of a differential constant Dc and the differential parameter D (Delta=(Pc)(P)+(Dc)(D)). At step 104, it is determined whether a rate of change of the valve position due to the valve position change Delta determined at the step 102 would be less than the allowable angular velocity of the valve Vv. If not, then at step 106 the change in the valve position of the control valve 74 is set to the maximum allowable valve position change Delta_{MAX}, which is equal to the product of the allowable angular velocity of the valve Vv and the update time interval between which the data associated with the fluid level 44 is read from the guided wave level sensor 66 (Delta_{MAX}=(Vv)(Update Interval)). If at the step 104 it is determined that the rate of change of the valve position due to the valve position change Delta determined at the step 102 is indeed less than the allowable angular velocity of the valve Vv, then at step 108 the valve position of the control valve 74 is updated by the valve position change Delta determined at the step 102. Alternatively, if the step 100 was executed, then at the step 108 the valve position of the control valve 74 is updated to remain unchanged, that is, updated by zero degrees. Alternatively, if the step 106 was executed, then at the step 108 the valve position of the control valve 74 is updated by the maximum allowable valve position change Delta_{MAX} of the control valve 74.

In an exemplary embodiment, at the step 108, to update the valve position of the control valve 74 by the valve position change Delta determined at the step 102, the electronic controller 68 sends one or more signals corresponding to the valve position change Delta to the electric actuator 72, which then opens, further opens, or further closes the control valve 74 by the valve position change Delta (or a value based thereupon). In an exemplary embodiment, at the step 108, to update the valve position of the control valve 74 by the maximum allowable valve position change Delta_{MAX} of the control valve 74, the electronic controller 68 sends one or more signals corresponding to the maximum allowable valve position change Delta_{MAX} to the electric actuator 72, which then opens, further opens, or further closes the control valve 74 by the maximum allowable valve position change Delta_{MAX} (or a value based thereupon).

As noted above, in several exemplary embodiments, the method 92 is repeatedly executed during the above-described operation of the apparatus 60. In an exemplary embodiment, the method 92 is executed upon the reading of data from the guided wave level sensor 66, the read data being associated with the fluid level 44 measured by the guided wave level sensor 66 at the end of one updated time interval. The execution of the method 92 is then repeated upon the reading of data from the guided wave level sensor 66, the read data being associated with the fluid level 44 measured by the guided wave level sensor 66 at the end of the next updated time interval; in several exemplary embodiments, this repeated execution of the method 92 continues during the operation of the apparatus 60. As a result, in several exemplary embodiments, the fluid level 44 is continuously, or nearly continuously, monitored and controlled by the apparatus 60.

As described above, the execution of the method 92 is based on the proportional constant Pc and the differential constant Dc. In an exemplary embodiment, each of the proportional constant Pc and the differential constant Dc is based on at least the diameter of the cylindrical wall 12h of the mud-gas separator vessel 62. In an exemplary embodiment, when the diameter of the cylindrical wall 12h is about 6 feet, the proportional constant Pc is 5 or another value, and the differential constant Dc is 15 or another value. In several exemplary embodiments, when the diameter of the cylindrical wall 12h is either about 5 feet or about 4 feet, the proportional constant Pc is 5 or another value, and the differential constant Dc is 15 or another value.

As described above, the execution of the method 92 is based on the proportional fluctuation constant Pf and the differential fluctuation constant Df. In an exemplary embodiment, the proportional fluctuation constant Pf is about 0.1. In an exemplary embodiment, the differential fluctuation constant Df is about 0.25. In an exemplary embodiment, the proportional fluctuation constant Pf and the differential fluctuation constant Df is about 0.1 and about 0.25, respectively. The execution of the step 98, and in particular the employment of the proportional fluctuation constant Pf and the differential fluctuation constant Df at the step 98, prevents the electric actuator 72 from having to make small or otherwise negligible adjustments to the valve position of the control valve 74, ensuring that only meaningful adjustments to the valve position are made, as necessary, during the above-described operation of the apparatus 60.

As described above, the execution of the method 92 is based on the set fluid level used in the determination at the step 94, which set fluid level is the desired value for the fluid level 44. In an exemplary embodiment, the set fluid level used in the determination at the step 94 is based on at least the diameter of the cylindrical wall 12h of the mud-gas separator vessel 62. In an exemplary embodiment, when the diameter of the cylindrical wall 12h is about 6 feet, the set fluid level is about 50 inches or another value. In several exemplary embodiments, when the diameter of the cylindrical wall 12h is either about 5 feet or about 4 feet, the set fluid level is about 50 inches or another value.

In several exemplary embodiments, in addition to the proportional constant Pc, the differential constant Dc, the proportional fluctuation constant Pf, the differential fluctuation constant Df, and the set fluid level, the execution of the method 92 may be based on one or more other parameters including, for example, one or both of the following parameters: the flow rate of the multiphase flow entering the internal region 12d in, for example, gallons per minute; and the density of the multiphase flow in, for example, pounds per gallon.

In an exemplary embodiment, during the execution of the method 92, the proportional constant Pc is about 5, the differential constant Dc is about 15, the proportional fluctuation constant Pf is about 0.1, the differential fluctuation constant Df is about 0.25, the diameter of the cylindrical wall 12h is about 6 feet, the flow rate of the multiphase flow entering the internal region 12d is about 1000 gallons per minute, the set fluid level is about 50 inches, and the density of the multiphase flow is about 16 pounds per gallon.

In several exemplary embodiments, the execution of the method 92 during the operation of the apparatus 60 provides intelligent system control, or active control, of the fluid level 44 in the apparatus 60, thereby eliminating the need to unnecessarily increase the mud leg 48 and allowing the mud-gas separator vessel 62 to operate more efficiently and separate more multiphase flow.

In several exemplary embodiments, the execution of the method 92 during the operation of the apparatus 60 permits the mud leg 48 to be within a predetermined range that is generally equal to a vertical height h of the level sensor housing assembly 64 (FIG. 13), thereby allowing the mud-gas separator vessel 62 to operate more efficiently and separate more multiphase flow; to achieve maintaining the mud leg 48 within this predetermined range, the set fluid level used in the determination at the step 94 may be located somewhere along the vertical height h, such as midway along the vertical height h. In several exemplary embodiments, the execution of the method 92 during the operation of the apparatus 60 permits the mud leg 48 to be within a predetermined range that is generally equal to a vertical distance defined by the level sensor housing assembly 64, such as, for example, the vertical height h of the level sensor housing assembly 64, the length of the tubular member 80, the vertical distance between the isolation valves 82 and 84, or the vertical distance between the fittings 76 and 78; to achieve maintaining the mud leg 48 within this predetermined range, the set fluid level used in the determination at the step 94 may be located somewhere along the vertical distance defined by the level sensor housing assembly 64, such as midway along the vertical distance defined by the level sensor housing assembly 64.

In an exemplary embodiment, during the above-described operation of the apparatus 60 including the above-described execution of the method 92, the mud-gas separator 62 may experience a “kick” situation, during which an increased amount of gas material flows into the internal region 12d of the mud-gas separator 62. The increased amount of gas material forces more of the slurry (i.e., the solid and liquid materials collected in the mud-gas separator vessel 62) to flow out of the internal region 12d, via the outlet 12c, and through the control valve 74, thereby rapidly reducing the fluid level 44 and thus the mud leg 48. However, the operation of the apparatus 60, including the execution of the method 92, automatically responds to the kick situation by accelerating the closing of the control valve 74 to maintain the mud leg 48 in a predetermined range, thereby maintaining the liquid seal that prevents vent gas carry under. In an exemplary embodiment, the predetermined range of the mud leg 48 maintained by the operation of the apparatus 60, including the execution of the method 92, is generally equal to a vertical distance defined by the level sensor housing assembly 64, such as, for example, the vertical height h of the level sensor housing assembly 64, the length of the tubular member 80, the vertical distance between the isolation valves 82 and 84, or the vertical distance between the fittings 76 and 78.

In an exemplary embodiment, during the above-described operation of the apparatus 60 including the above-described execution of the method 92, the fluid level 44 may begin to rapidly rise, causing the mud leg 48 to rapidly rise. This rapid rise in the fluid level 44, and thus the mud leg 48, may occur due to one or more reasons such as, for example, flow blockage or a clog in a fluid line located downstream of the control valve 74, or a rapid increase in the flow rate of the multiphase flow traveling into the internal region 12d. However, the operation of the apparatus 60, including the execution of the method 92, automatically responds to the rapid rise of the fluid level 44 by accelerating the opening of the control valve 74 to maintain the mud leg 48 in a predetermined range, thereby maintaining the separation performance of the mud-gas separator vessel 62. In an exemplary embodiment, the predetermined range of the mud leg 48 maintained by the operation of the apparatus 60, including the execution of the method 92, is generally equal to a vertical distance defined by the level sensor housing assembly 64, such as, for example, the vertical height h of the level sensor housing assembly 64, the length of the tubular member 80, the vertical distance between the isolation valves 82 and 84, or the vertical distance between the fittings 76 and 78.

In several exemplary embodiments, the method 92 may be employed to automatically control the control valve 26 in a manner substantially similar to the above-described manner by which the method 92 is employed to automatically control the control valve 74.

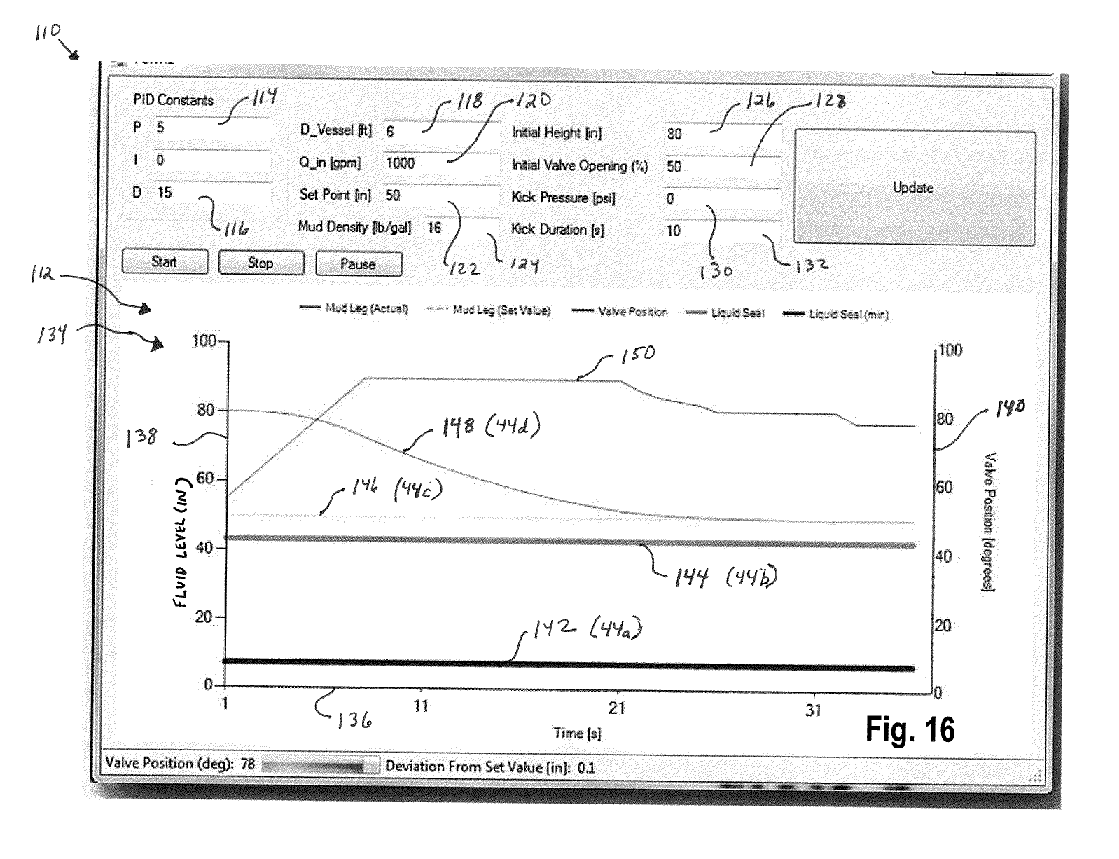

In an exemplary experimental embodiment, FIG. 16 illustrates a user interface 110 displayed on at least a portion of a display screen. In several exemplary experimental embodiments, the user interface 110 is part of a simulation program developed to fine tune and verify the performance of the algorithm employed in the method 92 of FIG. 15. In several exemplary experimental embodiments, the real-life behavior of an exemplary embodiment of the control valve 74 was modeled and embedded into the simulation program of which the user interface 110 is a part. As shown in FIG. 16, the user interface 110 includes an output 112 and input fields 114, 116, 118, 120, 122, 124, 126, 128, 130, 132, and 134. The proportional constant Pc is inputted in the field 114. The differential constant Dc is inputted in the field 116. The diameter of the cylindrical wall 12h is inputted in the field 118. The flow rate of the multiphase flow entering the internal region 12d is inputted in the field 120. The set fluid level to be used in the determination at the step 94 is inputted in the field 122; this set fluid level is the desired value for the fluid level 44. The density of the multiphase flow entering the internal region 12d is inputted in the field 124. In several exemplary embodiments, at least for the purpose of executing the simulation program of which the user interface 110 is a part, the initial height of the fluid level 44 is inputted in the field 126, the initial open percentage of the control valve 74 (the initial degree to which the control valve 74 is open) is inputted in the field 128 (0% if the valve 74 is completely closed, 100% if the valve 74 is completely open), a kick situation pressure is inputted in the field 130, and the kick situation duration is inputted in the field 132.

As shown in FIG. 16, the output 112 includes a chart 134. The chart 134 includes a horizontal axis 136 that indicates duration of time in, for example, seconds. In several exemplary embodiments, the duration of time indicated by the horizontal axis 136 represents the duration of time of operation of the apparatus 60, or at least the duration of time of the repeated execution of the method 92. The chart 134 further includes on either side thereof parallel-spaced vertical axes 138 and 140. The vertical axis 138 indicates the fluid level 44 in, for example, inches. The vertical axis 140 indicates the open position of the control valve 74 in, for example, degrees (0 degrees if the valve 74 is completely closed, 100 degrees if the valve 74 is completely open).

The chart 134 displays five data series, namely data series 142, 144, 146, 148, and 150. The data series 142 indicates a value of the fluid level 44 necessary to provide a minimum fluid seal. The data series 142 is a horizontal line, indicating that the minimum fluid seal is constant across the duration of time. In an exemplary embodiment, the fluid level 44 necessary to provide a minimum fluid seal is located slightly above, or is slightly higher than the vertical position of, the downward-facing end 12o of the vertically-extending segment 12n that is located near the drain outlet 32; this vertical location is referred to by the reference numeral 44a in FIG. 8.

The data series 144 indicates a value of the fluid level 44 necessary to provide a conservatively safe fluid seal. The data series 144 is a horizontal line, indicating that the conservative safe fluid seal is constant across the duration of time. In an exemplary embodiment, the fluid level 44 necessary to provide the conservatively safe fluid seal is located along the nominal center line of the horizontally-extending segment 121; this vertical location is referred to by the reference numeral 44b in FIG. 8.

The data series 146 indicates the set fluid level used in the determination at the step 94; this set fluid level is the desired value for the fluid level 44. The data series 146 is a horizontal line, indicating that the set fluid level used in the determination at the step 94 is constant across the duration of time. In an exemplary embodiment, the set fluid level used in the determination at the step 94 is located above, or is higher than the vertical position of, the vertical location 44b; this vertical location of the set fluid level is referred to by the reference 44c in FIG. 8.

The data series 148 indicates the actual fluid level 44, as measured by the guide wave level sensor 66. The data series 148 is not horizontal, but changes over time, indicating that the actual fluid level 44 varies over time. In an exemplary experimental embodiment, the vertical location of the actual fluid level 44 at a time of 1 second, as indicated by the chart 134 in the FIG. 16, is referred to by the reference numeral 44d in FIG. 8.

The data series 150 indicates the degree to which the control valve 74 is open. The data series 150 is not horizontal, but changes over time, indicating that the degree to which the control valve 74 is open varies over time.

As shown in FIG. 16, in an exemplary experimental embodiment, at a time of 1 second, the actual fluid level 44 indicated by the data series 148 is above the set fluid level indicated by the data series 146, potentially reducing the separation performance of the mud-gas separator vessel 62. However, the apparatus 60, due to the execution of the method 92, automatically detects this relatively high fluid level 44 and automatically responds to the relatively high fluid level 44 by accelerating the opening of the control valve 74 to a partially open valve position above 80 degrees, maintaining this partially open valve position for a period of time (until an elapsed time of about 21 seconds), and then automatically closing the control valve 74 to a partially open valve position of about 80 degrees, and subsequently to less than 80 degrees, when the actual fluid level 44 indicated by the data series 148 is about equal to the set fluid level indicated by the data series 146.

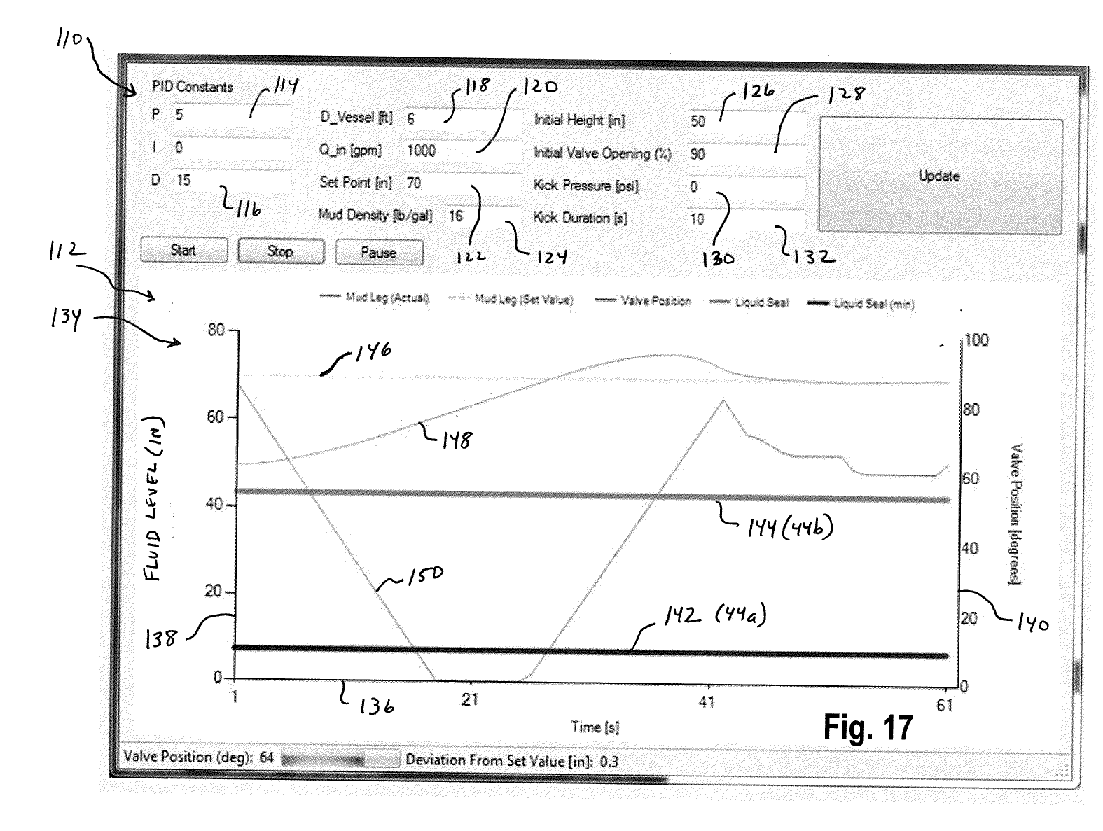

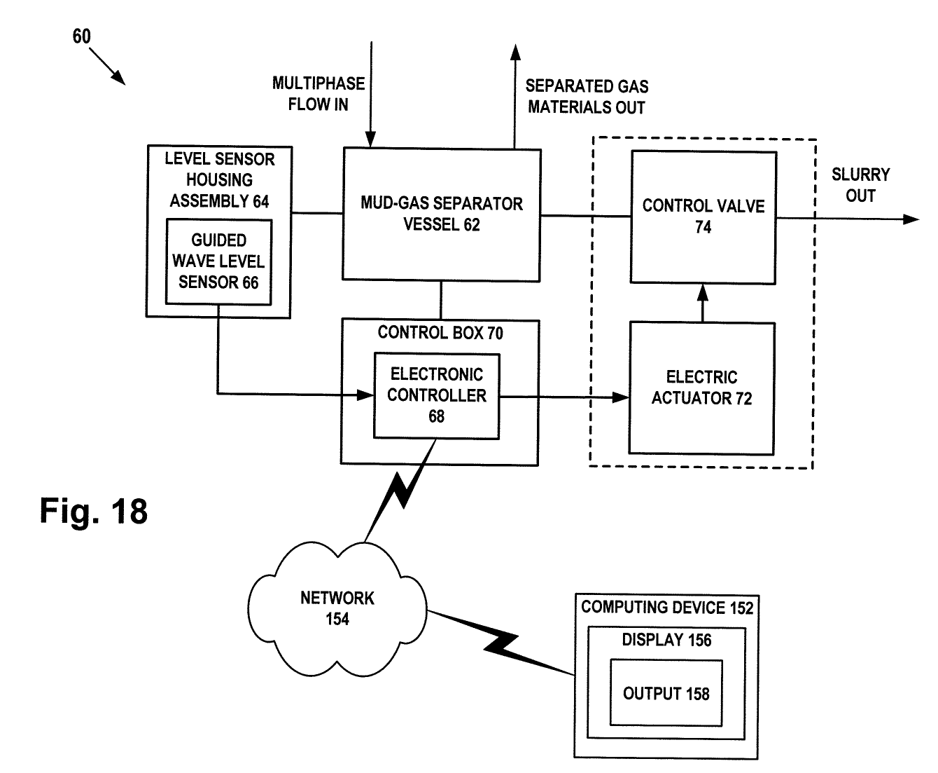

As shown in FIG. 17, in an exemplary experimental embodiment, at a time of 1 second, the actual fluid level 44 indicated by the data series 148 is below the set fluid level indicated by the data series 146, potentially increasing the risk of breaking the liquid seal provided by the slurry collected in the internal region 12d. However, the apparatus 60, due to the execution of the method 92, automatically detects this relatively low fluid level 44 and automatically responds to the relatively low fluid level 44 by accelerating the closing of the control valve 74 to a completely or fully closed valve position, maintaining this completely or fully closed valve position for a period of time (until an elapsed time of about 23-26 seconds), automatically opening the control valve 74 to a partially open valve position of about 80 degrees, and then automatically closing the control valve 74 to a partially open valve position of about 60 degrees, or slightly higher than 60 degrees, when the actual fluid level 44 indicated by the data series 148 is about equal to the set fluid level indicated by the data series 146.

Although FIG. 16 illustrates an exemplary experimental embodiment of the user interface 110 that is part of a simulation program developed to fine tune and verify the performance of the algorithm employed in the method 92 of FIG. 15, in several exemplary embodiments, the method 92 includes displaying an output on a display screen that is similar to the embodiment of the output 112 shown in FIG. 16, that is, when the fluid level 44 is rapidly rising and the apparatus 60 automatically responds by accelerating the opening of the control valve 74; in several exemplary embodiments, the electronic controller 68, and/or another computing device in communication with the electronic controller 68, is programmed to display this similar output during the execution of the method 92.

Similarly, although FIG. 17 illustrates an exemplary experimental embodiment of the user interface 110 that is part of a simulation program developed to fine tune and verify the performance of the algorithm employed in the method 92 of FIG. 15, in several exemplary embodiments, the method 92 includes displaying an output on a display screen that is similar to the embodiment of the output 112 shown in FIG. 17, that is, when the fluid level 44 is rapidly decreasing and the apparatus 60 automatically responds by accelerating the closing of the control valve 74; in several exemplary embodiments, the electronic controller 68, and/or another computing device in communication with the electronic controller 68, is programmed to display this similar output during the execution of the method 92.

In several exemplary embodiments, the electronic controller 68 includes one or more processors, a non-transitory computer readable medium operably coupled to the one or more processors, and a plurality of instructions (or computer program(s)) stored on the non-transitory computer readable medium, the instructions or program(s) being accessible to, and executable by, the one or more processors; in several exemplary embodiments, the one or more processors of the electronic controller 68 execute the plurality of instructions (or computer program(s)) to repeatedly execute at least the method 92 during the operation of the apparatus 60.

In an exemplary embodiment, as illustrated in FIG. 18 with continuing reference to FIGS. 1-17, the apparatus 60 includes a computing device 152 in communication with the electronic controller 68 via a network 154. The computing device 152 includes a display 156 on which output 158 is configured to be displayed, the output 158 being similar to the output 112 except that the respective data series displayed on the output 158 (which are equivalent to the data series 142, 144, 146, 148, and 150) indicate the different fluid levels and the valve position of the control valve 74 during the actual operation of the apparatus 60 (rather than a simulation). In an exemplary embodiment, the computing device 152 is located at the site at which the mud-gas separator vessel 62 is located. In an exemplary embodiment, the computing device 152 is remotely located from the mud-gas separator vessel 62. As a result, the computing device 152 permits the apparatus 60 to be remotely monitored. In an exemplary embodiment, the computing device 152 is a part of the electronic controller 68.

In an exemplary embodiment, the computing device 152 executes a program having a user interface that is similar to the user interface 110, except that the respective input fields that are part of the user interface executed on the computing device 152 (which are equivalent to at least the fields 114-124) are used to modify the automatic operation the apparatus 60 (rather than a simulation), and the respective data series displayed on the output 158 (which are equivalent to the data series 142, 144, 146, 148, and 150) indicate the different fluid levels and the valve position of the control valve 74 during the actual operation of the apparatus 60 (rather than a simulation). In an exemplary embodiment, the computing device 152 is located at the site at which the mud-gas separator vessel 62 is located. In an exemplary embodiment, the computing device 152 is remotely located from the mud-gas separator vessel 62. As a result, the computing device 152 permits the apparatus 60 to be remotely monitored, and further permits the operation of the apparatus 60 to be remotely modified by inputting one or more different values in one or more of the respective input fields that are equivalent to at least the fields 114-124.

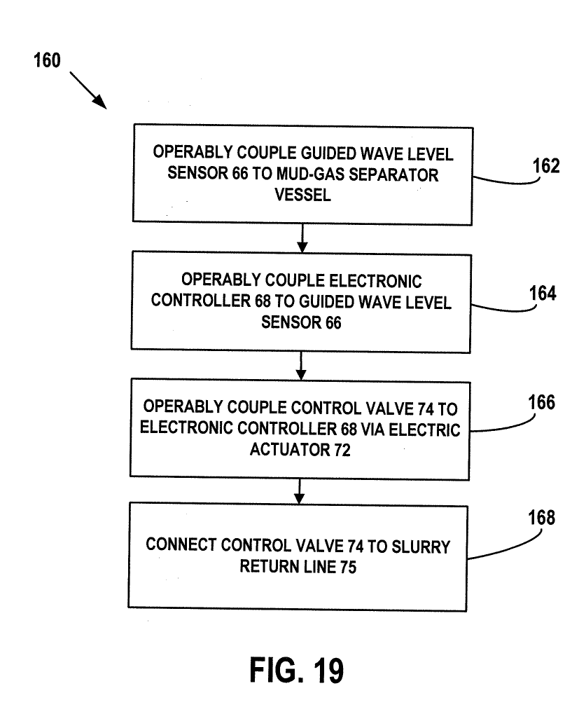

In an exemplary embodiment, as illustrated in FIG. 19 with continuing reference to FIGS. 1-18, a method of retrofitting a mud-gas separator apparatus is generally referred to by the reference numeral 160 and includes steps 162, 164, 166, and 168. At the step 162, the guided wave level sensor 66 is coupled to a mud-gas separator vessel. In an exemplary embodiment, the step 162 includes connecting the guided wave level sensor 66 to the level sensor housing assembly 64 in accordance with the foregoing, and connecting the level sensor housing 64 to the mud-gas separator vessel. At the step 164, the electronic controller 64 is operably coupled to the guided wave level sensor 66. At the step 166, the control valve 74 is operably coupled to the electronic controller 64. In an exemplary embodiment, the step 166 includes operably coupling the electric actuator 72 to the control valve 74, and operably coupling the electric actuator 72 to the electronic controller 68 so that the control valve 74 is operably coupled to the electronic controller 68 via the electric actuator 72. At the step 168, the control valve 74 is connected to the slurry return line 75.

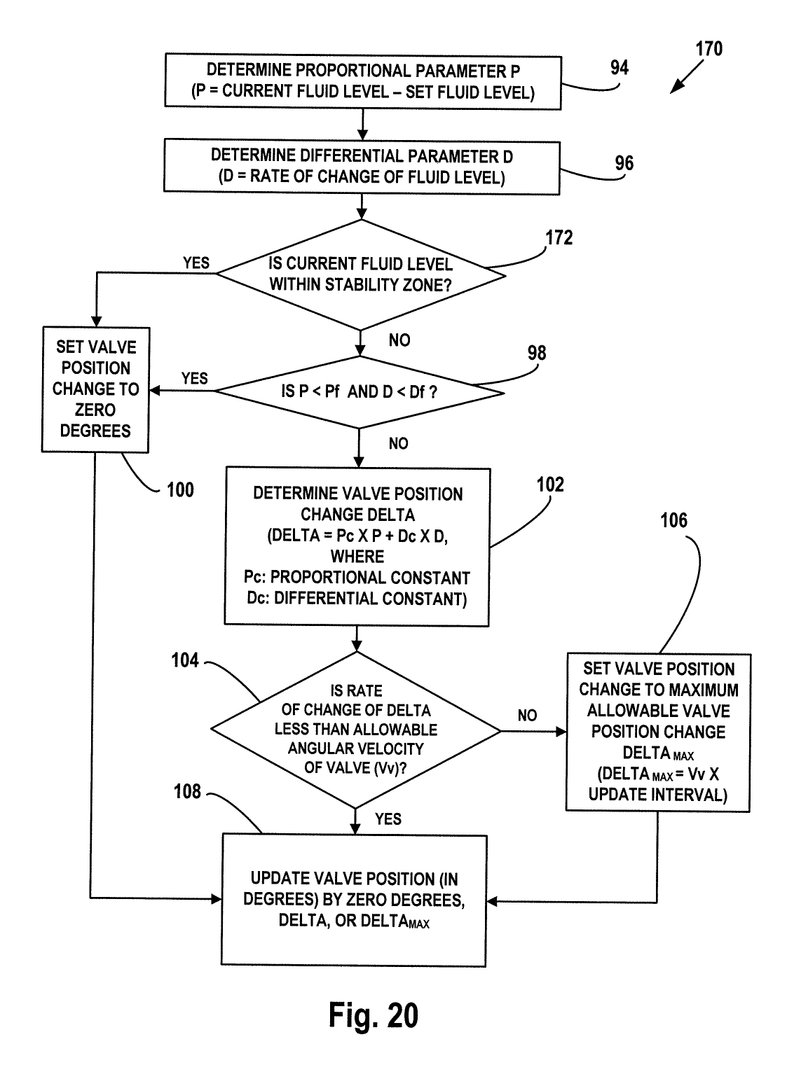

In an exemplary embodiment, as illustrated in FIG. 20 with continuing reference to FIGS. 1-19, a method of controlling the control valve 74, to actively control the fluid level 44 within the internal region 12d (or the mud leg 48 which is based on the fluid level 44), is generally referred to by the reference numeral 170. In several exemplary embodiments, the method 170 is repeatedly executed during the above-described operation of the apparatus 60.

In an exemplary embodiment, the method 170 is executed by the operation of the electronic controller 68, which is, includes, or is part of, a proportional derivative (PD) controller, as well as by the above-described operation of the guided wave level sensor 66, the electric actuator 72, and the control valve 74.

The method 170 includes all of the steps of the method 92, which steps are given the same reference numerals. The method 170 further includes a step 172, which in an exemplary embodiment is executed after the step 96 but before the step 98. At the step 172, it is determined whether the fluid level 44 is within a stability zone. If so, then the step 100 is executed. If it is determined at the step 172 that the fluid level is not within the stability zone, then the step 98 is executed. Except for the execution of the step 172, the execution of the method 170 is identical to the above-described execution of the method 92; therefore, the remainder of the execution of the method 170 will not be described in detail.

In several exemplary embodiments, the step 172 is executed before one or both of the steps 94 and 96. In several exemplary embodiments, the step 172 is executed after the step 98.

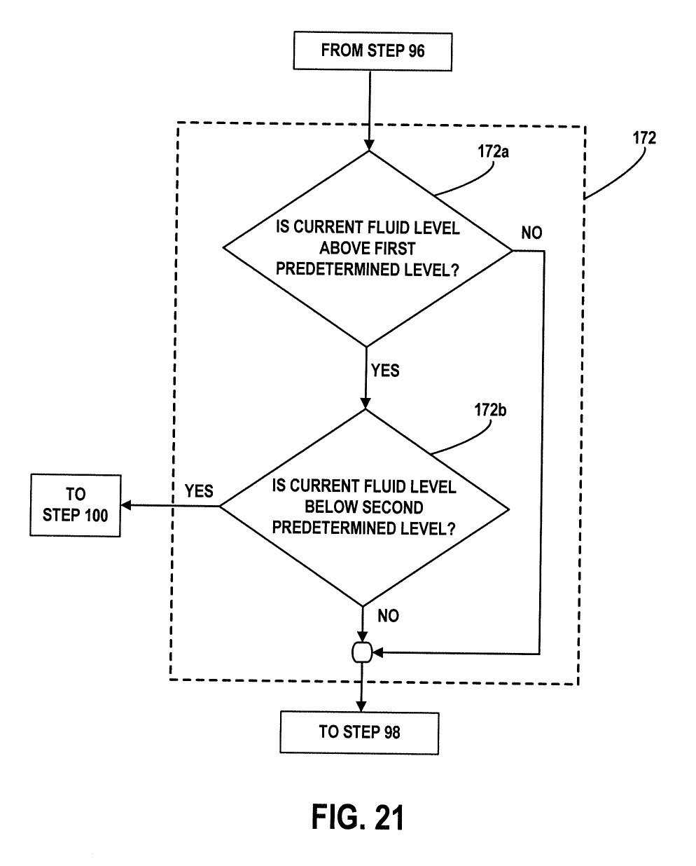

In an exemplary embodiment, as illustrated in FIG. 21 with continuing reference to FIGS. 1-20, the step 172 of the method 170 includes steps 172a and 172b. At the step 172a, it is determined whether the fluid level 44 is greater than about a first predetermined level (or a lower boundary fluid level). If it is determined at the step 172a that the fluid level 44 is not greater than about the first predetermined level, then it is determined that the fluid level 44 is not within the stability zone and the step 98 is executed. If it is determined at the step 172a that the fluid level 44 is greater than about the first predetermined level, then the step 172b is executed. At the step 172b, it is determined whether the fluid level 44 is less than about a second predetermined level (or an upper boundary fluid level). If so, then the fluid level 44 is determined to be within the stability zone that is defined between the first and second predetermined fluid levels employed at the steps 172a and 172b, respectively. Since the fluid level 44 is within the stability zone, the step 100 executed and thus the change in the valve position of the control valve 74 is set to zero (0) degrees, that is, the valve position of the control valve 74 is not to be changed. If it is determined at the step 172b that the fluid level 44 is above about the second predetermined level, then it is determined that the fluid level 44 is not within the stability zone and the step 98 is executed.

In an exemplary embodiment, the first predetermined level employed at the step 172a is the vertical location indicated by the reference numeral 44b in FIG. 8, or another vertical location. In an exemplary embodiment, the second predetermined level employed at the step 172b is the vertical location indicated by the reference numeral 44d in FIG. 8, or another vertical location. In an exemplary embodiment, the first and second predetermined levels employed at the steps 172a and 172b, respectively, are the vertical locations indicated by the reference numerals 44c and 44d, respectively.

In an exemplary embodiment, the execution of the method 170, and in particular the execution of the step 172 of the method 170, greatly reduces the duty cycle of the electric actuator 72. As a result, the useful operating lives of the electric actuator 72 and the control valve 74 are greatly prolonged.

In an exemplary embodiment, the execution of the method 170, and in particular the execution of the step 172 of the method 170, results in little or no change to the valve position of the control valve 74 so long as the flow rate of the multiphase flow traveling into the internal region 12d is generally constant. As a result, the useful operating lives of the electric actuator 72 and the control valve 74 are greatly prolonged.

In several exemplary embodiments, the method 170 may be employed to automatically control the control valve 26 in a manner substantially similar to the above-described manner by which the method 170 is employed to automatically control the control valve 74.

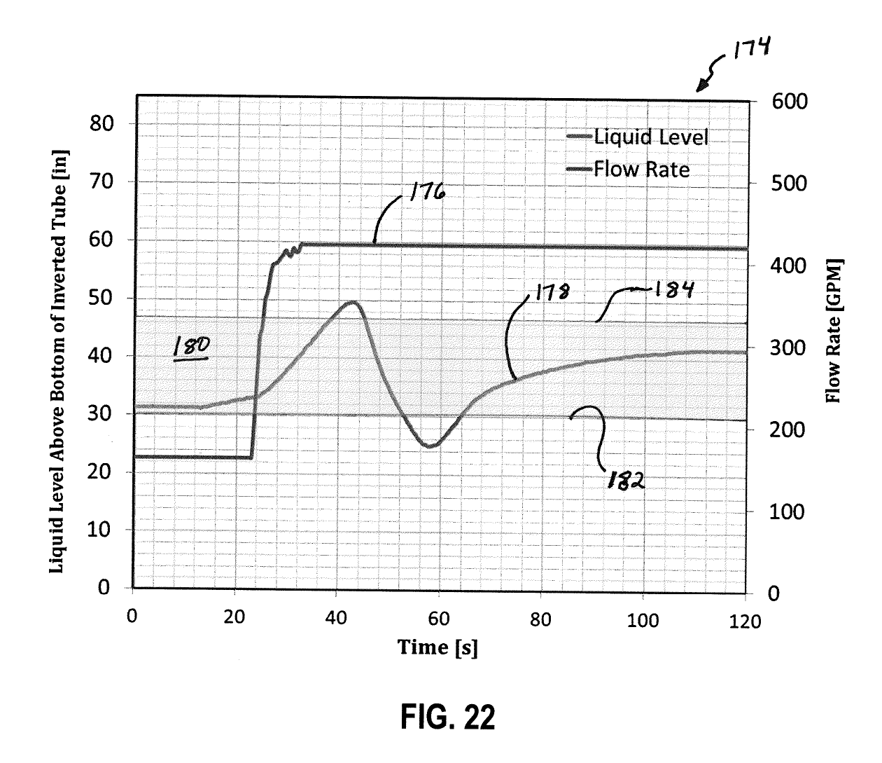

In an exemplary experimental embodiment, as illustrated in FIG. 22 with continuing reference to FIGS. 1-21, experimental testing was conducted using an exemplary experimental embodiment of the apparatus 60. During the experimental testing, the experimental exemplary embodiment of the apparatus 60 was operated in accordance with the above-described operation of the apparatus 60, and an experimental exemplary embodiment of the method 170 was executed during this operation. FIG. 22 includes a chart 174 describing the automatic response of the experimental embodiment of the apparatus 60 when the flow rate of the fluid traveling into the internal region 12d was quickly increased from about 160 gpm to about 420 gpm, as indicated by a data series 176 in the chart 174. The fluid level 44 over time is indicated by a data series 178. As shown in the chart 174, the mud-gas separator vessel 62 includes a stability zone 180. The stability zone 180 has a lower boundary fluid level 182, which is equal to the first predetermined level employed at the step 172a. The stability zone 180 also has an upper boundary fluid level 184, which is equal to the second predetermined level employed at the step 172b. The stability zone 180 extends between the fluid levels 182 and 184, and is unchanged over time. As shown in the chart 174, the lower boundary fluid level 182 was about 30 inches above the downward-facing end 12o of the vertically-extending segment 12n, and the upper boundary fluid level 184 was about 46 inches above the downward-facing end 12o.

As shown in the chart 174, in an exemplary experimental embodiment, at an initial flow rate of 160 gpm, the fluid level 44 within the mud-gas separator vessel 62 was within the stability zone 180, as indicated by the data series 178 from 0 to about 24 seconds. Beginning at about a time of 24 seconds, the flow rate was quickly increased from about 160 gpm to about 420 gpm at a time of about 30 seconds, causing the fluid level 44 to rise above the upper boundary fluid level 184 at around 40 seconds, outside of (or not within) the stability zone 180. In response, the automatic operation of the exemplary experimental embodiment of the apparatus 60, including the automatic execution of the exemplary experimental embodiment of the method 170, caused the fluid level 44 to drop to about 25 inches at about a time of 58 seconds, and then rise to a steady state fluid level 44 of about 42 inches at about a time of 120 seconds. This steady state fluid level 44 was within the stability zone 180. In an exemplary experimental embodiment, as shown in FIG. 22, the exemplary experimental embodiment of the apparatus 60 was able to stabilize in about 90 seconds in response to the quick increase of the flow rate from about 160 gpm to about 420 gpm, that is, the fluid level 44 was able to return to the stability zone 180 in about 90 seconds after the flow rate was increased to about 420 gpm.

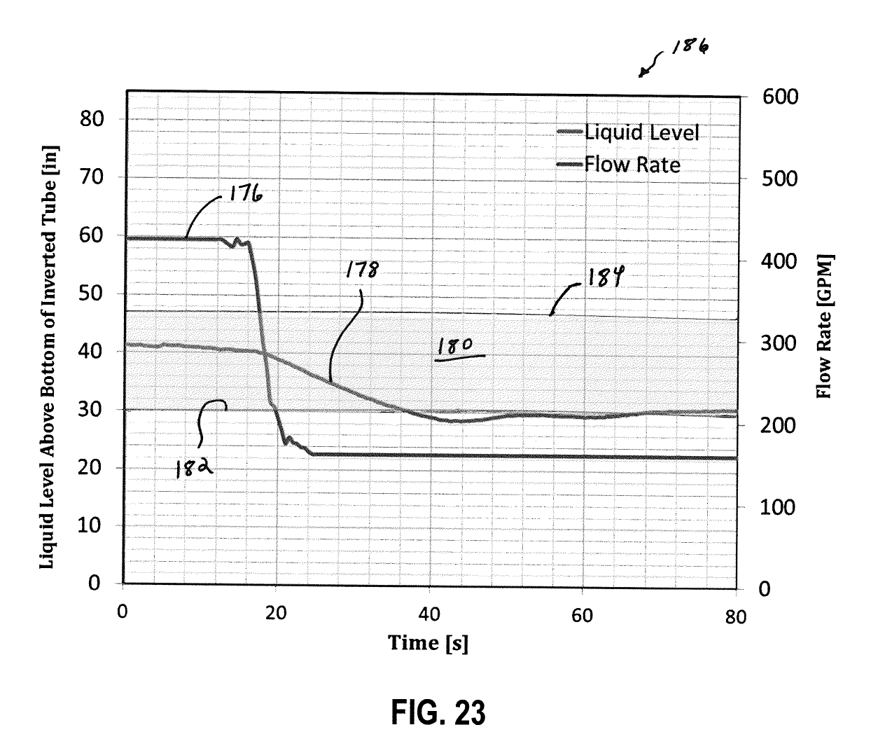

In an exemplary embodiment, as illustrated in FIG. 23 with continuing reference to FIGS. 1-22, a chart 186 describes the response of the experimental embodiment of the apparatus 60 when the flow rate of the fluid traveling into the internal region 12d was quickly decreased from about 420 gpm to about 160 gpm, as indicated by the data series 176 in the chart 186. As shown in the chart 186, at an initial flow rate of 420 gpm, the fluid level 44 within the mud-gas separator vessel 62 was within the stability zone 180, as indicated by the data series 178 from 0 to about 16 seconds. Beginning at a time of about 16 seconds, the flow rate was quickly decreased from about 420 gpm to about 160 gpm at a time of about 24 seconds, causing the fluid level 44 to drop below the lower boundary fluid level 182 at around 40 seconds, outside of the stability zone 180. In response, the automatic operation of the exemplary experimental embodiment of the apparatus 60, including the automatic execution of the exemplary experimental embodiment of the method 170, caused the fluid level 44 to increase to a steady state fluid level 44 of about 31 inches at about a time of 76 seconds. This steady state fluid level 44 was within the stability zone 180. In an exemplary experimental embodiment, as shown in FIG. 23, the exemplary experimental embodiment of the apparatus 60 was able to stabilize in about 52 seconds in response to the quick decrease of the flow rate from about 420 gpm to about 160 gpm, that is, the fluid level 44 was able to return to the stability zone 180 in about 52 seconds after the flow rate was decreased to about 160 gpm.

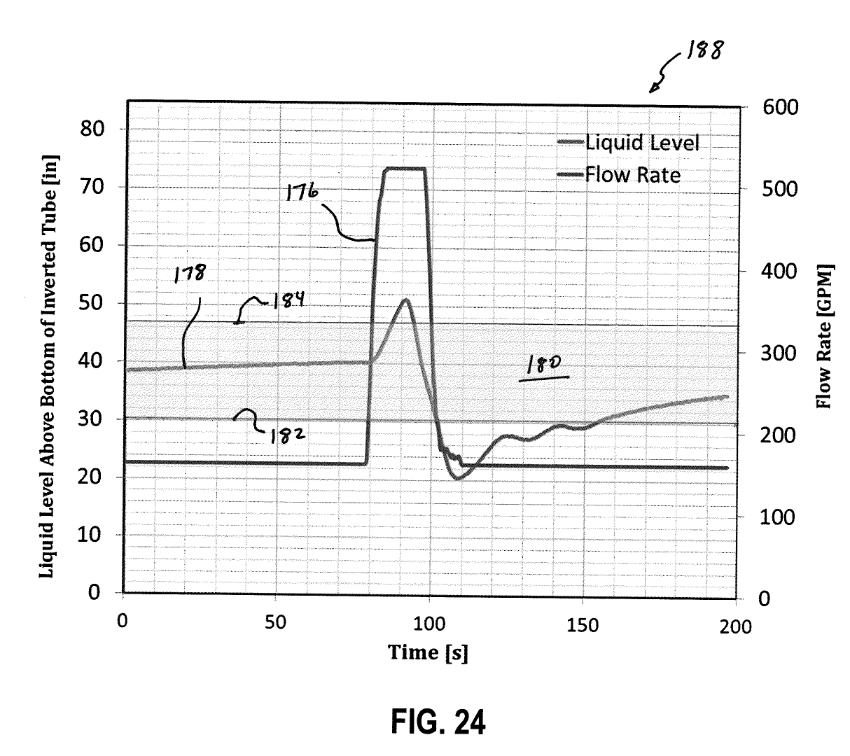

In an exemplary embodiment, as illustrated in FIG. 24 with continuing reference to FIGS. 1-23, a chart 188 describes the response of the experimental embodiment of the apparatus 60 when the apparatus 60 was subjected to a disturbance in which the flow rate of the fluid traveling into the internal region 12d was quickly increased from about 160 gpm to about 530 gpm, held at about 530 gpm for about 10-15 seconds, and then quickly decreased back down to about 160 gpm, as indicated by the data series 176 in the chart 188. As shown in the chart 188, at an initial flow rate of 160 gpm, the fluid level 44 within the mud-gas separator vessel 62 was within the stability zone 180, as indicated by the data series 178 from 0 to about 85 seconds. Beginning at a time of about 80 seconds, the flow rate was quickly increased from about 160 gpm to about 530 gpm at about a time of 85 seconds, causing the fluid level 44 to rise above the upper boundary fluid level 184 slightly thereafter, outside of the stability zone 180. The flow rate was held at about 530 gpm for about 10-15 seconds, and then was quickly decreased back down to about 160 gpm, causing a disturbance, or temporary spike, in the flow rate. In response to the disturbance, or temporary spike, in the flow rate, the automatic operation of the exemplary experimental embodiment of the apparatus 60, including the automatic execution of the exemplary experimental embodiment of the method 170, caused the fluid level 44 to drop to about 20 inches at about a time of 110 seconds, and then rise to a steady state fluid level 44 of about 35 inches at about a time of 200 seconds or slightly past 200 seconds (e.g., 205 seconds). This steady state fluid level 44 was within the stability zone 180. In an exemplary experimental embodiment, as shown in FIG. 22, the exemplary experimental embodiment of the apparatus 60 was able to stabilize in about 120 seconds in response to the disturbance in the flow rate, that is, the fluid level 44 was able to return to the stability zone 180 in about 120 seconds after the disturbance, or temporary spike, in the flow rate.

In several exemplary embodiments, a plurality of instructions, or computer program(s), are stored on a non-transitory computer readable medium, the instructions or computer program(s) being accessible to, and executable by, one or more processors. In several exemplary embodiments, the one or more processors execute the plurality of instructions (or computer program(s)) to repeatedly execute at least the method 92, or at least the method 170, during the operation of the apparatus 60. In several exemplary embodiments, the one or more processors are part of the electronic controller 68, the computing device 152, one or more other computing devices, or any combination thereof. In several exemplary embodiments, the non-transitory computer readable medium is part of the electronic controller 68, the computing device 152, one or more other computing devices, or any combination thereof.

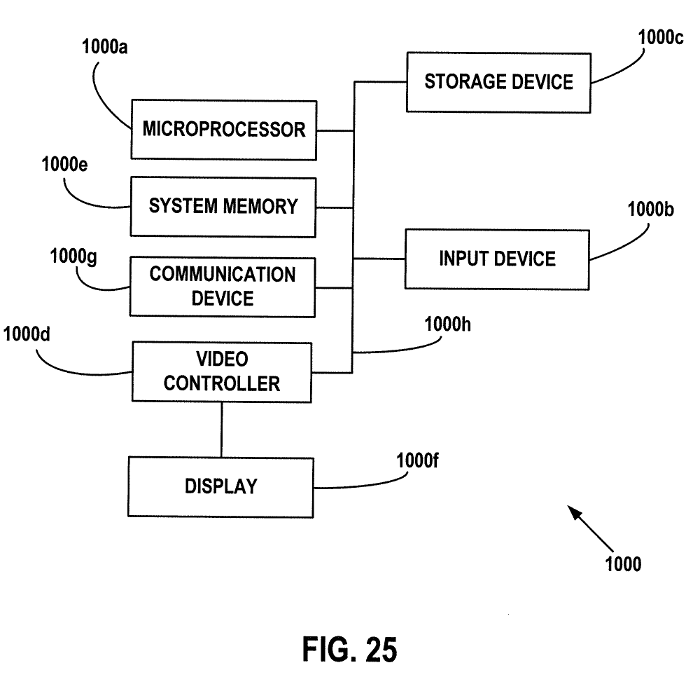

In an exemplary embodiment, as illustrated in FIG. 25 with continuing reference to FIGS. 1-24, an illustrative node 1000 for implementing one or more embodiments of one or more of the above-described networks, elements, methods and/or steps, and/or any combination thereof, is depicted. The node 1000 includes a microprocessor 1000a, an input device 1000b, a storage device 1000c, a video controller 1000d, a system memory 1000e, a display 1000f, and a communication device 1000g all interconnected by one or more buses 1000h. In several exemplary embodiments, the storage device 1000c may include a floppy drive, hard drive, CD-ROM, optical drive, any other form of storage device and/or any combination thereof. In several exemplary embodiments, the storage device 1000c may include, and/or be capable of receiving, a floppy disk, CD-ROM, DVD-ROM, or any other form of computer-readable medium that may contain executable instructions. In several exemplary embodiments, the communication device 1000g may include a modem, network card, or any other device to enable the node to communicate with other nodes. In several exemplary embodiments, any node represents a plurality of interconnected (whether by intranet or Internet) computer systems, including without limitation, personal computers, mainframes, PDAs, smartphones and cell phones.

In several exemplary embodiments, one or more of the components of the apparatus 10, 50, 52, or 60, such as one or more of the sensors 14, 52a, 52b, 52c, 52d, and 52e, the electronic controller 16, the control valve 26, the guided wave level sensor 66, the electronic controller 68, the electric actuator 72, the control valve 74, and the computing device 152, include at least the node 1000 and/or components thereof, and/or one or more nodes that are substantially similar to the node 1000 and/or components thereof. In several exemplary embodiments, one or more of the above-described components of the node 1000 and/or the apparatus 10, 50, 53, or 60, include respective pluralities of same components.

In several exemplary embodiments, a computer system typically includes at least hardware capable of executing machine readable instructions, as well as the software for executing acts (typically machine-readable instructions) that produce a desired result. In several exemplary embodiments, a computer system may include hybrids of hardware and software, as well as computer sub-systems.

In several exemplary embodiments, hardware generally includes at least processor-capable platforms, such as client-machines (also known as personal computers or servers), and hand-held processing devices (such as smart phones, tablet computers, personal digital assistants (PDAs), or personal computing devices (PCDs), for example). In several exemplary embodiments, hardware may include any physical device that is capable of storing machine-readable instructions, such as memory or other data storage devices. In several exemplary embodiments, other forms of hardware include hardware sub-systems, including transfer devices such as modems, modem cards, ports, and port cards, for example.

In several exemplary embodiments, software includes any machine code stored in any memory medium, such as RAM or ROM, and machine code stored on other devices (such as floppy disks, flash memory, or a CD ROM, for example). In several exemplary embodiments, software may include source or object code. In several exemplary embodiments, software encompasses any set of instructions capable of being executed on a node such as, for example, on a client machine or server.

In several exemplary embodiments, combinations of software and hardware could also be used for providing enhanced functionality and performance for certain embodiments of the present disclosure. In an exemplary embodiment, software functions may be directly manufactured into a silicon chip. Accordingly, it should be understood that combinations of hardware and software are also included within the definition of a computer system and are thus envisioned by the present disclosure as possible equivalent structures and equivalent methods.

In several exemplary embodiments, computer readable mediums include, for example, passive data storage, such as a random access memory (RAM) as well as semi-permanent data storage such as a compact disk read only memory (CD-ROM). One or more exemplary embodiments of the present disclosure may be embodied in the RAM of a computer to transform a standard computer into a new specific computing machine. In several exemplary embodiments, data structures are defined organizations of data that may enable an embodiment of the present disclosure. In an exemplary embodiment, a data structure may provide an organization of data, or an organization of executable code.

In several exemplary embodiments, any networks and/or one or more portions thereof, may be designed to work on any specific architecture. In an exemplary embodiment, one or more portions of any networks may be executed on a single computer, local area networks, client-server networks, wide area networks, internets, hand-held and other portable and wireless devices and networks.

In several exemplary embodiments, a database may be any standard or proprietary database software. In several exemplary embodiments, the database may have fields, records, data, and other database elements that may be associated through database specific software. In several exemplary embodiments, data may be mapped. In several exemplary embodiments, mapping is the process of associating one data entry with another data entry. In an exemplary embodiment, the data contained in the location of a character file can be mapped to a field in a second table. In several exemplary embodiments, the physical location of the database is not limiting, and the database may be distributed. In an exemplary embodiment, the database may exist remotely from the server, and run on a separate platform. In an exemplary embodiment, the database may be accessible across the Internet. In several exemplary embodiments, more than one database may be implemented.

In several exemplary embodiments, a plurality of instructions stored on a computer readable medium may be executed by one or more processors to cause the one or more processors to carry out or implement in whole or in part the above-described operation of each of the above-described exemplary embodiments of the mud-gas separator apparatus 10, 50, 53, or 60, the method 54, the method 92, the method 170, and/or any combination thereof. In several exemplary embodiments, such a processor may include one or more of the microprocessor 1000a, any processor(s) that are part of the components of the mud-gas separator apparatus 10, 50, 53, or 60, and/or any combination thereof, and such a computer readable medium may be distributed among one or more components of the mud-gas separator apparatus 10, 50, 53, or 60. In several exemplary embodiments, such a processor may execute the plurality of instructions in connection with a virtual computer system. In several exemplary embodiments, such a plurality of instructions may communicate directly with the one or more processors, and/or may interact with one or more operating systems, middleware, firmware, other applications, and/or any combination thereof, to cause the one or more processors to execute the instructions.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose. Terms such as “left” and right”, “front” and “rear”, “above” and “below” and the like are used as words of convenience to provide reference points and are not to be construed as limiting terms.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

1. An apparatus, comprising:
a mud-gas separator vessel adapted to receive a multiphase flow and separate gas materials therefrom, the mud-gas separator vessel defining an internal region in which a slurry is adapted to be collected, the slurry defining a fluid level within the internal region;
at least one sensor operably coupled to the mud-gas separator vessel and adapted to measure the fluid level when the slurry is collected in the internal region;
an electronic controller in communication with the at least one sensor and adapted to receive from the at least one sensor measurement data associated with the measurement of the fluid level; and
a control valve in communication with the electronic controller and adapted to control discharge of the slurry out of the mud-gas separator vessel;
wherein the electronic controller is adapted to automatically control the control valve based on the measurement data received from the at least one sensor and thus actively control the fluid level within the internal region using the control valve.

2. The apparatus of claim 1, wherein the control valve comprises an electric actuator and a rotary control valve operably coupled thereto.

3. The apparatus of claim 1, wherein the at least one sensor comprises a guided wave level sensor, the guided wave level sensor comprising a probe; and
wherein the apparatus further comprises a level sensor housing assembly connected to the mud-gas separator vessel, the level sensor housing assembly comprising a tubular member within which at least a portion of the probe extends.

4. The apparatus of claim 3, wherein the level sensor housing assembly further comprises:
first and second fittings between which the tubular member extends; and
first and second isolation valves connected to the first and second fittings, respectively, and to the mud-gas separator vessel;
wherein the tubular member is spaced from the mud-gas separator vessel; and
wherein the guided wave level sensor is connected to the second fitting and the probe extends through the second fitting and at least into the tubular member.

5. The apparatus of claim 1, wherein the electronic controller comprises:
one or more processors;
a non-transitory computer readable medium operably coupled to the one or more processors; and
a plurality of instructions stored on the non-transitory computer readable medium and executable by the one or more processors, the plurality of instructions comprising instructions that cause the one or more processors to automatically control the control valve based on the measurement data.

6. The apparatus of claim 5, wherein the instructions that cause the one or more processors to automatically control the control valve comprise:
instructions that cause the one or more processors to automatically further close the control valve in response to determining that the fluid level is decreasing too rapidly; and
instructions that cause the one or more processors to automatically open, or further open, the control valve in response to determining that the fluid level is increasing too rapidly.

7. The apparatus of claim 5, wherein the instructions that cause the one or more processors to automatically control the control valve comprise:
instructions that cause the one or more processors to determine that the fluid level is not within a stability zone; and
instructions that cause the one or more processors to automatically adjust a valve position of the control valve in response to determining that the fluid level is not within the stability zone.

8. The apparatus of claim 5, wherein the instructions that cause the one or more processors to automatically control the control valve comprise:
instructions that cause the one or more processors to determine a proportional parameter; and
instructions that cause the one or more processors to determine a differential parameter.

9. The apparatus of claim 8, wherein the instructions that cause the one or more processors to automatically control the control valve further comprise:
instructions that cause the one or more processors to determine a valve position change based on the proportional and differential parameters if either:
the proportional parameter is not less than a proportional fluctuation constant; or
the differential parameter is not less than a differential fluctuation constant; and
instructions that cause the one or more processors to set a change in a valve position of the control valve to zero degrees if:
the proportional parameter is less than the proportional fluctuation constant; and
the differential parameter is less than the differential fluctuation constant.

10. The apparatus of claim 9, wherein the instructions that cause the one or more processors to automatically control the control valve further comprise instructions that cause the one or more processors to determine a maximum allowable valve position change if a rate of change of the valve position due to the valve position change based on the proportional and differential parameters would not be less than an allowable angular velocity of the control valve.

11. The apparatus of claim 10, wherein the instructions that cause the one or more processors to automatically control the control valve further comprise:
instructions that cause the one or more processors to update the valve position of the control valve by the valve position change based on the proportional and differential parameters if:
the rate of change of the valve position due to the valve position change based on the proportional and differential parameters would be less than the allowable angular velocity of the control valve; and
either:
the proportional parameter is not less than the proportional fluctuation constant, or
the differential parameter is not less than the differential fluctuation constant;
instructions that cause the one or more processors to update the valve position of the control valve by the maximum allowable valve position change if:
the rate of change of the valve position due to the valve position change based on the proportional and differential parameters would not be less than the allowable angular velocity of the control valve; and
either:
the proportional parameter is not less than the proportional fluctuation constant, or
the differential parameter is not less than the differential fluctuation constant; and
instructions that cause the one or more processors to update the valve position of the control valve by zero degrees if:
the proportional parameter is less than a proportional fluctuation constant; and
the differential parameter is less than a differential fluctuation constant.

12. A method of actively controlling a fluid level in an internal region defined by a mud-gas separator vessel, the fluid level being defined by a slurry collected within the internal region, the method comprising:
automatically measuring, using at least one sensor, the fluid level in the internal region;
automatically transmitting, using the at least one sensor, measurement data to an electronic controller, the measurement data being associated with the measurement of the fluid level defined by the slurry; and
automatically controlling, using the electronic controller, a control valve based on the measurement data;
wherein the automatic control of the control valve by the electronic controller automatically controls discharge of the slurry out of the mud-gas separator vessel and thus actively controls the fluid level.

13. The method of claim 12, wherein automatically controlling the control valve comprises:
automatically further closing the control valve in response to determining that the fluid level is decreasing too rapidly; and
automatically opening, or further opening, the control valve in response to determining that the fluid level is increasing too rapidly.

14. The method of claim 12, wherein automatically controlling the control valve comprises:
automatically determining that the fluid level is not within a stability zone; and
automatically adjusting the valve position of the control valve in response to determining that the fluid level is not within the stability zone.

15. The method of claim 12, wherein automatically controlling the control valve comprises:
automatically determining a proportional parameter; and
automatically determining a differential parameter.

16. The method of claim 15, wherein automatically controlling the control valve further comprises:
automatically determining a valve position change based on the proportional and differential parameters if either:
the proportional parameter is not less than a proportional fluctuation constant; or
the differential parameter is not less than a differential fluctuation constant; and
automatically setting a change in a valve position of the control valve to zero degrees if:
the proportional parameter is less than the proportional fluctuation constant; and
the differential parameter is less than the differential fluctuation constant.

17. The method of claim 16, wherein automatically controlling the control valve further comprises automatically determining a maximum allowable valve position change if a rate of change of the valve position due to the valve position change based on the proportional and differential parameters would not be less than an allowable angular velocity of the control valve.

18. The method of claim 17, wherein automatically controlling the control valve further comprises:
automatically updating the valve position of the control valve by the valve position change based on the proportional and differential parameters if:
the rate of change of the valve position due to the valve position change based on the proportional and differential parameters would be less than the allowable angular velocity of the control valve; and
either:
the proportional parameter is not less than the proportional fluctuation constant, or
the differential parameter is not less than the differential fluctuation constant;
automatically updating the valve position of the control valve by the maximum allowable valve position change if:
the rate of change of the valve position due to the valve position change based on the proportional and differential parameters would not be less than the allowable angular velocity of the control valve; and
either:
the proportional parameter is not less than the proportional fluctuation constant, or
the differential parameter is not less than the differential fluctuation constant; and
automatically updating the valve position of the control valve by zero degrees if:
the proportional parameter is less than the proportional fluctuation constant; and
the differential parameter is less than the differential fluctuation constant.

19-24. (canceled)

25. A kit for actively controlling a fluid level within an internal region defined by a mud-gas separator vessel, the fluid level being defined by a slurry collected within the internal region, the kit comprising:
at least one sensor adapted to be operably coupled to the mud-gas separator vessel, and to measure the fluid level when the slurry is collected in the internal region;
an electronic controller adapted to be in communication with the at least one sensor, and to receive from the at least one sensor measurement data associated with the measurement of the fluid level; and
a control valve adapted to be in communication with the electronic controller, and to control discharge of the slurry out of the mud-gas separator vessel;
wherein the electronic controller is adapted to automatically control the control valve based on the measurement data received from the at least one sensor and thus is adapted to actively control the fluid level within the internal region using the control valve.

26. The kit of claim 25, wherein the at least one sensor comprises a guided wave level sensor, the guided wave level sensor comprising a probe; and
wherein the kit further comprises a level sensor housing assembly adapted to be connected to the mud-gas separator vessel, the level sensor housing assembly comprising a tubular member within which at least a portion of the probe extends.

27-34. (canceled)
